(19) United States
(12) Patent Application Publication
(10) Pub. No.: US 2025/0261956 A1
(43) Pub. Date: Aug. 21, 2025

(54) DEVICES AND METHODS FOR ENHANCED ASPIRATION-INDUCED THROMBECTOMY

(71) Applicant: Imperative Care, Inc., Campbell, CA (US)

(72) Inventors: Miranda M. Ray, San Jose, CA (US); Yi Yang, San Francisco, CA (US); Mathurin Boutte, San Jose, CA (US)

(21) Appl. No.: 19/033,337

(22) Filed: Jan. 21, 2025

Related U.S. Application Data
(63) Continuation of application No. PCT/US2023/070626, filed on Jul. 20, 2023.
(60) Provisional application No. 63/391,472, filed on Jul. 22, 2022.

(51) Int. Cl. A61B 17/22; A61B 17/00

(52) U.S. Cl. CPC A61B 17/22; A61B 17/00234; A61B 2017/00238; A61B 2017/00544; A61B 2017/00561; A61B 2017/22079; A61B 2217/005

(57) ABSTRACT

Described herein are various embodiments of valve systems that comprise a compressible region that is configured to reduce vacuum at a first port of the valve while substantially maintaining vacuum at a second port of the valve. Further described herein is an improved aspiration canister configured to maintain vacuum at a second vacuum line while a first vacuum line is disconnected from the canister.

### INCORPORATION BY REFERENCE

This application is a continuation application of International Patent Application No. PCT/US2023/070626 filed Jul. 20, 2023, which claims the priority benefit of U.S. Provisional Patent Application No. 63/391,472, filed Jul. 22, 2022, the entirety of each of which is hereby incorporated by reference herein. All publications and patent applications mentioned in this specification are herein incorporated by reference in their entirety, as if each individual publication or patent application was specifically and individually indicated to be incorporated by reference in its entirety.

### FIELD

This disclosure relates generally to the field of thrombectomy, and more specifically to the field of aspiration based thrombectomy. Described herein are hemostasis valves for maintaining vacuum during guide catheter removal.

### BACKGROUND

Thrombotic restrictions and occlusions within a patient's blood vessels are a significant medical problem and often require intervention to remove these restrictions and blockages to restore health to patients. While applicable to a wide range of vascular applications in both the arterial and venous systems, including a variety of small vessels such as arterial blockages in the neuro vasculature (ischemic stroke), the following background illuminates the problems primarily through the example of patients suffering with Pulmonary Embolisms.

Venous thromboembolic disease (VTE) is a worldwide crisis. There are over 10 million cases of deep vein thrombosis (DVT) and pulmonary embolism (PE) diagnosed globally per year, with 1 million cases occurring in the United States and over 700,000 in France, Italy, Germany, Spain, Sweden, and the United Kingdom combined each year. There are approximately 60,000 to 100,000 deaths from PE in the United States each year. DVT and PE are part of the same continuum of disease, with over 95% of emboli originating in the lower extremities. When PE occurs, the severity depends on the embolic burden and its effect on the right ventricle as well as underlying cardiopulmonary comorbidities. Death can result from the acute increase in pulmonary artery (PA) pressure with increased right ventricular (RV) afterload and dysfunction.

Patients with high-risk pulmonary embolism (PE) and other ischemic conditions were treated primarily with thrombolytic therapy delivered systemically or more locally through Catheter Directed Thrombolytics. These approaches result in multiple catheterization lab visits, lengthy hospital stays and often lead to bleeding complications. Newer approaches to both PE and ischemic stroke include single session thrombectomy (aspiration of the clot) without the use of thrombolytics. These thrombectomy treatments include positioning the distal end of an aspiration catheter adjacent the clot and applying suction in an effort to aspirate the clot through the catheter and into a cannister outside of the sterile field.

Conventional thrombectomy procedures use a vacuum pressure syringe (i.e., lock the plunger for a negative pressure vacuum) on the guide catheter during an aspiration procedure. However, if the clot shears at the guide catheter during retraction, the vacuum inside of the rotating hemostasis valve (RHV) needs to be maintained to not lose the clot while the guide catheter is removed to clear the clot from tip. If the RHV is opened, blood flow will push the clot off the tip of the guide catheter creating a new stroke in the patient. Splitting the aspiration so it connects with the pump improves clot retention on the guide but does not solve the problem entirely.

Accordingly, a new way is needed to maintain vacuum on the guide catheter while removing the aspiration catheter.

### SUMMARY

One aspect of the present disclosure is directed to an aspiration system for a thrombectomy procedure. The aspiration system can include a source of vacuum; an aspiration canister including a first port, a second port, and a third port. The third port can be in fluid communication with the source of vacuum, The aspiration system can include a first aspiration line including a proximal end removably connected to the first port, wherein the first aspiration line is configured to be in fluid communication with a first catheter to provide a first fluid flow path between a distal end of the first catheter and the aspiration canister. The aspiration system can include a second aspiration line including a proximal end removably connected to the second port, wherein the second aspiration line is configured to be in fluid communication with a second catheter to provide a second fluid flow path between a distal end of the second catheter and the aspiration canister, the second catheter having an inner diameter smaller than the first catheter. The aspiration system can include a flow reducer positioned along the first fluid flow path and configured to reduce a fluid flow rate along the first fluid flow path proximal to the flow reducer when vacuum is applied to the first aspiration line. The aspiration system can provide uninterrupted vacuum to the first and second aspiration lines when the first and second aspiration lines are connected to the first and second ports. The aspiration system can provide vacuum to the first aspiration line at or above a minimum vacuum pressure threshold for maintaining a clot engaged to a distal tip of the first catheter.

In any of the embodiments described herein, the first port can include a first self-sealing port configured to seal the aspiration canister when the first aspiration line is not connected to the first self-sealing port, and the second port can include a second self-sealing port configured to seal the second self-sealing port when the second aspiration line is not connected to the second self-sealing port.

In any of the embodiments described herein, the flow reducer can be positioned at a distal region of the first aspiration line.

In any of the embodiments described herein, the flow reducer can be connected to the first port.

In any of the embodiments described herein, the minimum vacuum pressure threshold can be −14 inHg.

In any of the embodiments described herein, the flow reducer can include a main body including a first end, a second end, and a cavity; a fluid flow path segment extending from the first end to the second end; a restrictor including a hole; and one or more baffles extending from an inner surface of the main body. The one or more baffles each can include one or more slots.

In any of the embodiments described herein, the one or more slots can be offset relative to one another to increase a length of the fluid flow path segment.

In any of the embodiments described herein, the one or more slots can be spaced by about 1 mm from each other.

In any of the embodiments described herein, the one or more baffles can form an angle of about 90° with respect to the inner surface of the main body.

In any of the embodiments described herein, the first aspiration line can include a clot retrieval device.

In any of the embodiments described herein, the clot retrieval device can include a distal port and a proximal port, the distal port having a diameter larger than a diameter of the proximal port.

In any of the embodiments described herein, the first aspiration line can include a first tubing section distal to the clot retrieval device and a second tubing section proximal to the clot retrieval device, the second tubing section can have an inner diameter smaller than an inner diameter of the first tubing section to reduce the fluid flow rate through the second tubing section.

In any of the embodiments described herein, the flow reducer can be positioned proximal to the clot retrieval device.

In any of the embodiments described herein, the flow reducer can be connected to a proximal port of the clot retrieval device.

In any of the embodiments described herein, the flow reducer can be configured to reduce the fluid flow rate by at least about 27%.

In any of the embodiments described herein, the flow reducer can be configured to reduce the fluid flow rate by at least about 31%.

In any of the embodiments described herein, the flow reducer can be configured to reduce the fluid flow rate by at least about 35%.

In any of the embodiments described herein, the flow reducer can be configured to reduce the fluid flow rate by at least about 46%.

In any of the embodiments described herein, the flow reducer can be configured to reduce the fluid flow rate by between 25% and 50%.

In any of the embodiments described herein, the flow reducer can be configured to maintain the fluid flow rate below a maximum threshold value, wherein the maximum threshold value is between 140 ml/min and 200 mL/min.

In any of the embodiments described herein, the aspiration system can include the first catheter, wherein the first catheter can be coupled to a hemostasis valve. The aspiration system can be configured to provide vacuum to the first aspiration line at or above the minimum vacuum pressure threshold for maintaining the clot engaged to the distal tip of the first catheter while the hemostasis valve is open.

Another aspect of the present disclosure is related to a method of performing a thrombectomy procedure. The method can include connecting a distal end of a first aspiration line to a first catheter to establish a first fluid flow path between the first catheter and an aspiration canister. A proximal end of the first aspiration line can be coupled to a first port of the aspiration canister. A flow reducer can be positioned along the first fluid flow path. The method can include connecting a distal end of a second aspiration line to a second catheter to establish a second fluid flow path between the second catheter and the aspiration canister. A proximal end of the second aspiration line can be coupled to a second port of the aspiration canister. The method can include advancing the first catheter and the second catheter though a vasculature of a patient to a target vascular site having a clot while the second catheter is positioned within a lumen of the first catheter; applying vacuum to the aspiration canister to apply vacuum to the first catheter and the second catheter along the first fluid flow path and the second fluid flow path; and retracting the second catheter out of a hemostasis valve of the first catheter while maintaining a vacuum pressure in the first catheter at or greater than a minimum vacuum pressure threshold for maintaining the clot engaged to a distal tip of the first catheter. The flow reducer can reduce a fluid flow rate along the first fluid flow path proximal to the flow reducer.

In any of the embodiments described herein, the minimum vacuum pressure threshold can be about −14 inHg.

In any of the embodiments described herein, maintaining the vacuum pressure in the first catheter at or greater than the minimum vacuum pressure threshold can include maintaining the vacuum pressure between −14 inHg and −28 inHg.

In any of the embodiments described herein, the method can include closing the hemostasis valve after retracting the second catheter out of the hemostasis valve and closing the second aspiration line. The flow reducer can be configured to retain the fluid flow rate along the first fluid flow path proximal to the flow reducer at or below a maximum threshold value when the hemostasis valve and second aspiration line are closed.

In any of the embodiments described herein, the maximum threshold value of the fluid flow rate can be between 140 ml/min and 200 mL/min.

In any of the embodiments described herein, the first aspiration line can include a clot retrieval device.

In any of the embodiments described herein, the clot retrieval device can include a distal port and a proximal port, the distal port having a larger diameter larger than a diameter of the proximal port.

In any of the embodiments described herein, the first aspiration line includes a first tubing section distal to the clot retrieval device and a second tubing section proximal to the clot retrieval device, wherein the second tubing section has an inner diameter smaller than an inner diameter of the first tubing section to reduce the fluid flow rate through the second tubing section.

In any of the embodiments described herein, the flow reducer can be positioned proximal to the clot retrieval device.

In any of the embodiments described herein, the flow reducer can be connected to a proximal port of the clot retrieval device.

In any of the embodiments described herein, the flow reducer can be positioned along a distal section of the first aspiration line.

In any of the embodiments described herein, the flow reducer can be connected to the first port.

In any of the embodiments described herein, the flow reducer can include a plurality of baffles, each baffle including a slot to facilitate fluid flow therethrough.

In any of the embodiments described herein, the slots of adjacent baffles are offset relative to one another to increase a length of the fluid flow path within the flow reducer.

In any of the embodiments described herein, the flow reducer can reduce the fluid flow rate by at least about 27%.

In any of the embodiments described herein, the flow reducer can reduce the fluid flow rate by at least about 31%.

In any of the embodiments described herein, the flow reducer can reduce the fluid flow rate by at least about 35%.

In any of the embodiments described herein, the flow reducer can reduce the fluid flow rate by at least about 46%.

In any of the embodiments described herein, the flow reducer can reduce the fluid flow rate by between 25% and 50%.

In any of the embodiments described herein, each of the first port and the second port can include a self-sealing port.

In any of the embodiments described herein, the aspiration canister can include a third port coupled to a vacuum source, wherein the third port has a different structure than the first port and the second port.

One aspect of the present disclosure is directed to a valve system comprising one or more of the following: a first port configured to be fluidly connected to a proximal end of a first catheter; a second port configured to be fluidly connected to a source of vacuum; a third port configured to receive a second catheter therethrough, such that the second catheter is configured to be coaxially disposed in a lumen of the first catheter; and a compressible region between the first port and the third port and distal to the second port.

In any of the embodiments described herein, application of compression to the compressible region reduces vacuum at the third port and substantially maintains or increases vacuum at the first port.

In any of the embodiments described herein, the valve further comprises a first vacuum line fluidly connected to the second port and a second vacuum line connected to the third port.

In any of the embodiments described herein, the first vacuum line and the second vacuum line are fluidly connected to a common vacuum line, wherein the common vacuum line is configured to fluidly connect the second port and the third port to the source of vacuum.

In any of the embodiments described herein, the valve further comprises a split connector that is configured to connect the first and second vacuum lines to the common vacuum line.

In any of the embodiments described herein, the compressible region comprises an elastic material comprising an embedded fiber braid.

In any of the embodiments described herein, the compressible region comprises silicone, polyurethane, or a combination thereof.

Another aspect of the present disclosure is directed to a method of adjusting vacuum at a valve system. The method comprises one or more of the following steps: connecting a proximal end of a first catheter to a first port of the valve system; connecting a second port of the valve system to a source of vacuum; disposing a second catheter through a third port of the valve system, such that the second catheter is configured to be coaxially disposed in a lumen of the first catheter; and applying compression to a compressible region of the valve system.

In any of the embodiments described herein, the compressible region is between the first port and the third port and distal to the second port.

In any of the embodiments described herein, application of compression to the compressible region reduces vacuum at the third port and substantially maintains or increases vacuum at the first port.

In any of the embodiments described herein, the method further includes connecting a first vacuum line to the second port and a second vacuum line to the third port.

In any of the embodiments described herein, the method further includes connecting the first vacuum line and the second vacuum line to a common vacuum line; and connecting the common vacuum to the source of vacuum.

In any of the embodiments described herein, the method further includes connecting a split connector between the first and second vacuum lines and the common vacuum line.

In any of the embodiments described herein, the compressible region comprises an elastic material comprising an embedded fiber braid.

In any of the embodiments described herein, the compressible region comprises silicone, polyurethane, or a combination thereof.

Another aspect of the present disclosure is directed to an aspiration canister comprising: a reservoir; a lid configured to be coupled to the reservoir; and a valve disposed in the lid.

In any of the embodiments described herein, the valve is configured such that one or more vacuum lines are configured to be added or removed during a procedure. In any of the preceding embodiments, the valve is a needless valve.

Another aspect of the present disclosure is directed to a method of switching between aspiration lines during a thrombectomy procedure. The method comprises: coupling a first vacuum line to a first port disposed in a lid of an aspiration canister; coupling a second vacuum line to a second port disposed in the lid of the aspiration canister; and disconnecting the first vacuum line from the first port while not substantially losing vacuum at the second vacuum line.

In any of the embodiments described herein, one or both of the first port and the second port comprise a needless valve.

In any of the embodiments described herein, any of the steps are configured to be performed during active thrombectomy.

In any of the embodiments described herein, any of the steps are configured to be performed before active thrombectomy.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates an embodiment of a valve system.

FIG. 2 illustrates an embodiment of a split vacuum line.

FIG. 3 illustrates an embodiment of an aspiration system.

FIG. 4 illustrates an embodiment of a canister of an aspiration system.

FIGS. 5A-5C illustrate an embodiment of a canister of an aspiration system.

FIG. 6A illustrates an embodiment of a canister of an aspiration system with a cap secured to a port.

FIG. 6B illustrates the cap shown in FIG. 6A.

FIG. 7A illustrates an embodiment of a canister of an aspiration system with a flow reducer secured to a valve.

FIG. 7B illustrates an embodiment of a flow reducer.

FIG. 7C illustrates an exploded view of embodiment of a flow reducer with four baffles.

FIG. 7D illustrates a cross-section view of the flow reducer shown in FIG. 7C.

FIG. 7E illustrates an exploded view of an embodiment of a flow reducer with three baffles.

FIG. 7F illustrates a cross-section view of the flow reducer shown in FIG. 7E.

FIG. 7G illustrates an exploded view of an embodiment of a flow reducer with four baffles.

FIG. 7H illustrates an exploded view of an embodiment of a flow reducer with four baffles.

FIG. 7I illustrates an embodiment of a canister of an aspiration system with a flow reducer secured to a distal end of an aspiration line.

FIG. 8A illustrates a cross section view of an embodiment of a flow reducer.

FIG. 8B illustrates a partially transparent view of the flow reducer shown in FIG. 8A.

FIG. 8C illustrates a cross section view of an embodiment of a flow reducer with eight baffles.

FIG. 8D illustrates a partially transparent view of the flow reducer shown in FIG. 8C.

FIG. 8E illustrates a cross section view of another embodiment of a flow reducer with eight baffles.

FIG. 8F illustrates a partially transparent view of the flow reducer shown in FIG. 8E.

FIGS. 9A-9B illustrate an embodiment of a canister of an aspiration system with self-sealing valves.

FIG. 9C illustrates an embodiment of the sealing valve shown in FIGS. 9A-9B.

FIGS. 10A-10B illustrate an embodiment of an aspiration line with a clot retrieval device.

FIGS. 11A-11B illustrate an embodiment of an aspiration line with a clot retrieval device and connectors for a self-sealing port.

FIGS. 11C-11D illustrate embodiments of adapters for use with the aspiration lines shown in FIGS. 10A-11B.

FIG. 12 illustrates a first catheter and a second catheters inside the vasculature of a patient.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Any of the devices, structures, features, system, or methods described herein may be utilized in connection with any suitable aspiration system. For example, any embodiment described herein may be utilized in connection with any of the aspiration systems as described in U.S. Pat. Nos. 10,835,272; 11,065,018; 11,25,9821 or in U.S. patents application Ser. No. 17/036,258 or U.S. Ser. No. 17/857,598, each of which is hereby incorporated by reference herein.

### Modified Valve System

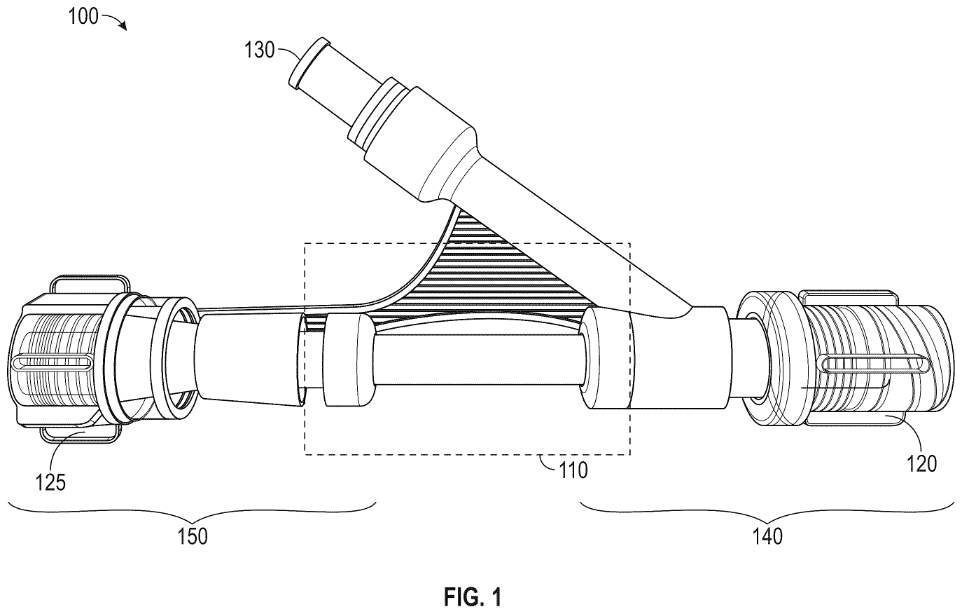

FIG. 1 shows a valve system 100. The valve system 100 may incorporate any suitable type of valve (e.g., a hemostasis valve or a rotating hemostasis valve) that may be utilized to allow passage of one or more catheter systems through the valve system 100 and/or to allow application of aspiration and/or vacuum pressure through the valve system 100 and/or any catheter systems passed therethrough. For example, the catheter systems may comprise one or more catheters (e.g., a guide catheter, aspirate catheter, guidewire, etc.) as described in any of the U.S. patents or patent applications discussed and incorporated by reference herein. A distal entry port 120 of the valve system 100 may be connected directly or indirectly to one or more catheter systems to be inserted into a patient.

In some instances, a guide catheter (or any other suitable catheter) may be connected directly or indirectly to distal entry port 120 while a secondary device (e.g., an aspiration catheter or any other suitable insertion device) may pass through a proximal entry port 125 and be inserted into the guide catheter through the distal entry port 120. An aspiration source may be coupled to the side port 130 to apply aspiration and/or vacuum pressure at least through the valve system 100 (e.g., the distal entry port 120) and to any fluidly connected catheter systems.

During retraction of the secondary device, concern must be taken to ensure that a loss of vacuum pressure does not occur at the distal entry port 120 or along any connected catheter systems. In some instances, any loss of vacuum pressure may result in dislodgement of a clot, thrombus, or other vascular occlusion that has been captured by a connected catheter system during an aspiration procedure. For example, if fluid communication between the aspiration source through the side port 130 and the distal entry port 120, then a clot held at the end of a catheter system connected to the distal entry port 120 may no longer remain engaged to the catheter system and may become pass back into a patient's vasculature. This would result in harm to the patient by creating an additional occlusion risk to the patient.

With reference to FIG. 1, the valve system 100 may include a modified region 110 configured to allow a healthcare practitioner to, manually or automatically, temporarily inhibit fluid communication between the proximal entry port 125 and the distal entry port 120. Temporarily isolating the proximal entry port 125 from fluid communication with the remainder of the valve system 100 may advantageously facilitate removal of a secondary device, as described herein, while minimizing any risk of loss of vacuum pressure through the distal entry port 120 and any related connected catheter systems.

In some instances, the modified region 110 may be compressible by a healthcare practitioner to manually shut off the distal entry port 120 of the valve system 100 when external compression is applied to the compressible region 110. Applying compression to the compressible region 110 of the valve system 100 may advantageously inhibit substantial loss of vacuum through side port 130 by isolating the valve system 100 into two regions: a proximal region 150 and a distal region 140. When the compressible region 110 is engaged, the distal region 140 of the valve system 100 may be configured to substantially maintain any present vacuum pressure even if the proximal region 150 of the valve system 100 experiences a substantial reduction in vacuum pressure (e.g., due to opening of the proximal entry port 125 to permit withdrawal of a secondary device). As shown in FIG. 1, compressible region 110 may comprise a resilient tube fluidly connecting the distal region 140 with the distal entry port 120 and the proximal region 150 with the distal entry port 125. The resilient tube may be compressible, while also having sufficient resilience and/or hoop strength to inhibit collapse of the tube during application of standard aspiration pressures used during an aspiration procedure. In some instances, the compressible region 110 may have sufficient burst strength to inhibit expansion during a high-pressured contrast injection procedure. For example, the compressible region 110 may comprise silicone, polyurethane, elastic materials comprising an embedded fiber braid, and/or any other material with sufficient properties.

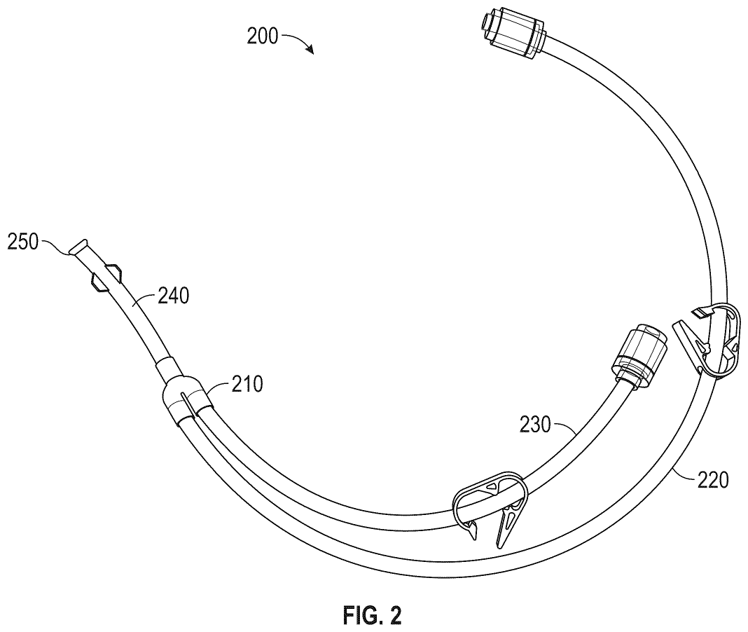

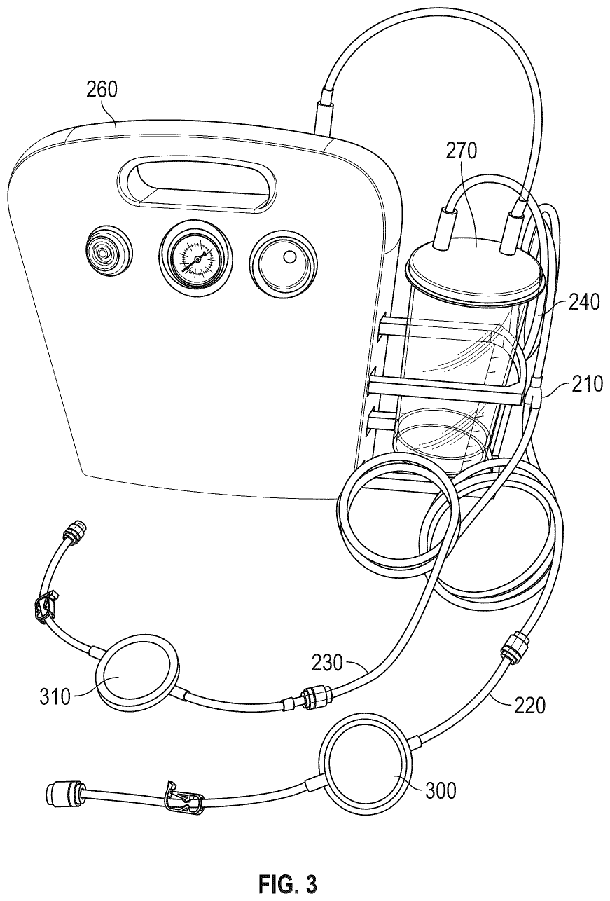

Alternatively, or additionally, the valve system 100 shown in FIG. 1 may be used in conjunction with a split vacuum line 200, as illustrated in FIG. 2, or alone to maintain vacuum on the guide catheter (or any other suitable catheter) while removing the guide catheter (or any other suitable catheter) with the clot still engaged at the tip. In some embodiments, the valve system 100 can be used with an aspiration system (e.g., the aspiration systems described with respect to FIGS. 3-12) to maintain vacuum on a catheter while removing the catheter with the clot still engaged at the tip.

As shown in FIGS. 2-3, a split aspiration or vacuum line 200 comprises a common vacuum line 240 split at connector 210 (e.g., Wye connector) into a first vacuum line 220 and a second vacuum line 230. Port 250 of common vacuum line 240 fluidly connects to the canister 270 of the aspiration pump 260, first vacuum line 220 fluidly connects to a valve system (e.g., a rotating hemostasis valve) on a proximal end of a first catheter (e.g., guide catheter), and second vacuum line 240 fluidly connects to a valve system (e.g., a rotating hemostasis valve) on a proximal end of a second catheter (e.g., procedure catheter, aspiration catheter, etc.) configured to be disposed in the first catheter. The split vacuum line 200 increases the potential vacuum source from a fixed volume RHV to a continuous pump system. The first vacuum line 220 can include a clot retrieving device 300 positioned between a distal end and a proximal end of the first vacuum line 220. The second vacuum line 230 can include a clot retrieving device 310 positioned between a distal end and a proximal end of the first vacuum line 230. The clot retrieving devices 300, 310 can each retain clots aspirated via the first and second catheters connected to the first and second vacuum lines 220, 230.

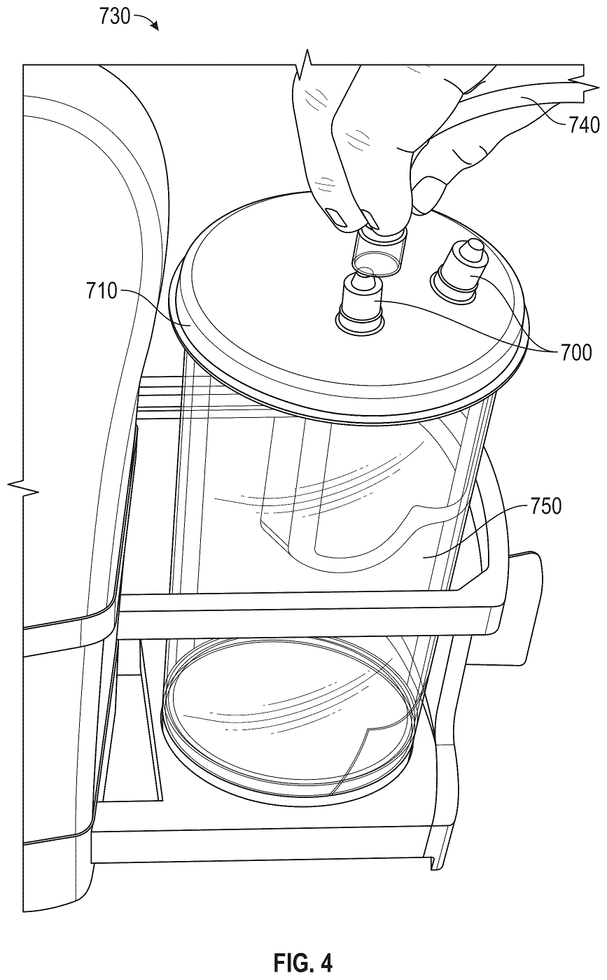

In some embodiments, as shown in FIG. 4, an aspiration canister 730 may comprise one or more closeable valves or ports 700 installed on the lid 710 coupled to a reservoir 750. The closeable port 700 enables a vacuum line 740 to be added and removed as needed. Each of the ports 700 is self-shutting or self-sealing, such that when the port 700 is not connected to a line 740, the port 700 is closed and vacuum pressure is substantially maintained or vacuum pressure is not substantially lost within the canister 730. In some instances, the canister 730 allows a physician to add additional vacuum lines or aspiration lines at any stage during a procedure. Any additional aspiration lines (e.g., a secondary line) may be fluidly connected to one of the closeable ports 700 at any stage prior to or during an aspiration procedure. In some instances, multiple aspiration lines may be added and/or removed during an aspiration procedure. For example, during an aspiration procedure, a healthcare practitioner may desire to include an additional aspiration line to provide additional aspiration through a secondary device as described herein (e.g., a secondary catheter or a guide catheter) by connecting a new aspiration line between one of the closeable ports 700 and the secondary device during the procedure. Aspiration through the secondary device may occur as an alternative or in addition to aspiration through a primary device (e.g., a first catheter). In contrast, ports that are not self-sealing may result in an unacceptable loss of pressure when a line is not connected to the port, preventing replacement or addition of an aspiration line during an aspiration procedure. The closeable port 700 may comprise any suitable closeable needless valve connector.

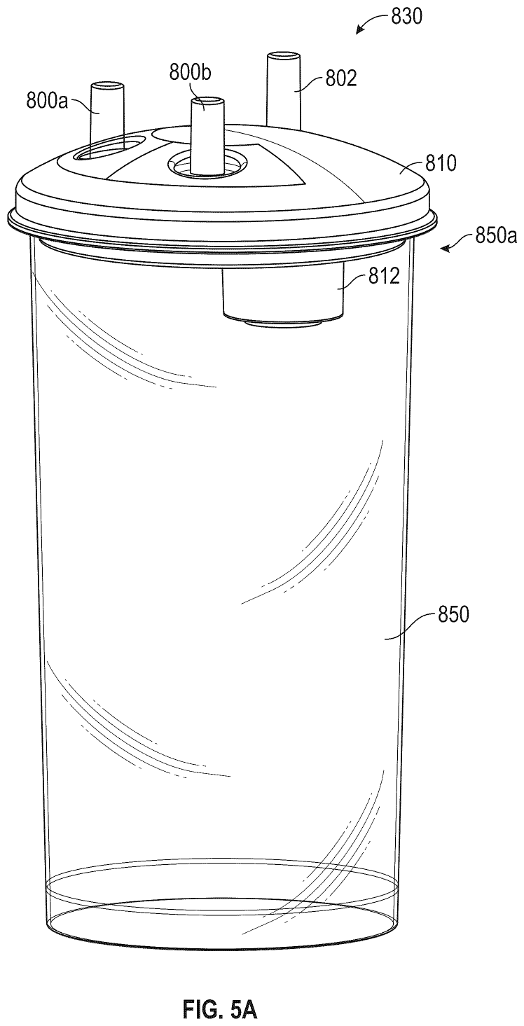

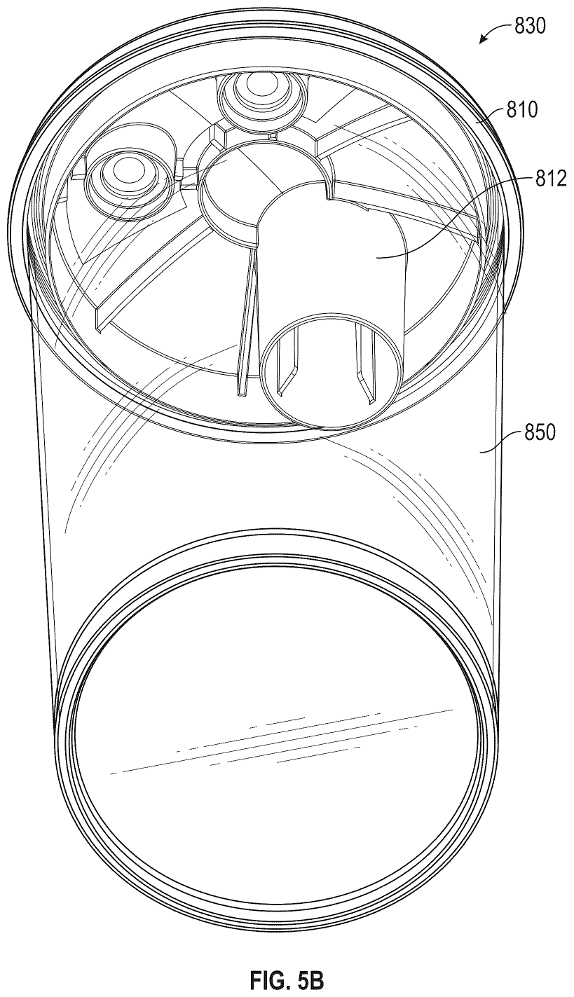

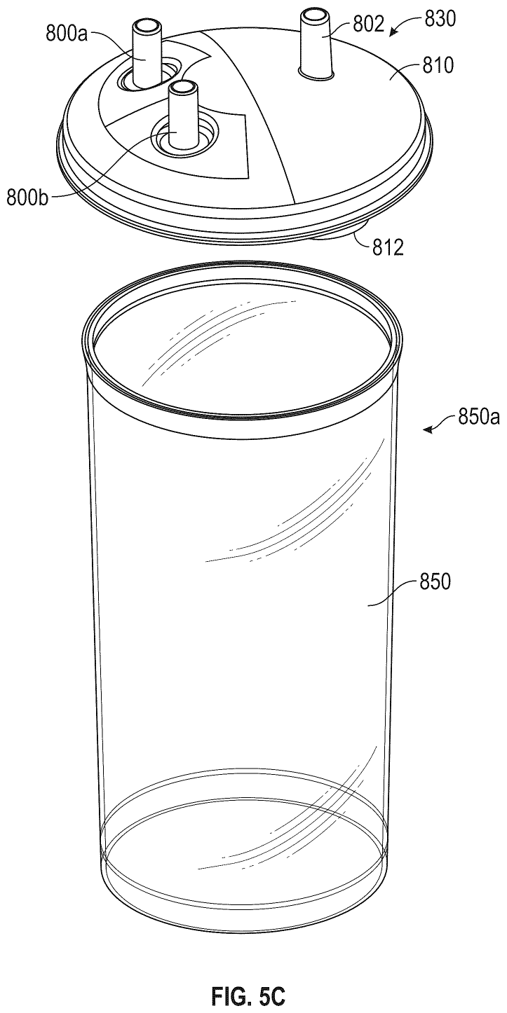

FIGS. 5A-5C show an embodiment of an aspiration canister including two valves or ports. The aspiration canister 830 can include a first valve or port 800a, a second valve or port 800b, and an aspiration port 802. The first port 800a, second port 800b, and aspiration port 802 can be positioned on a lid 810 which can removably secured to a reservoir 850 of the aspiration canister 830, as shown in FIG. 5C. The aspiration port 802 can be coupled (e.g., by a tube) to a source of vacuum, such as an aspiration pump, to provide vacuum to the aspiration canister 830 and connectors in fluid communication therewith. A vacuum line (not shown), which can include any of the same or similar features and functions as the vacuum line 740, can be secured to each of the first and second ports 800a, 800b. In some embodiments, a single vacuum line can be connected to the first port 800a or the second port 800b. In some embodiments, however, each of the first and second ports 800a, 800b can receive and secure a distinct vacuum line. This can beneficially allow physicians to add and/or remove vacuum lines based on a desired aspiration procedure. For example, while a first vacuum line is secured to the first port 800a, a second vacuum line can be secured to the second port 800b to allow aspiration through both the first and second vacuum lines. As described in further detail herein, each of the first and second vacuum lines can be coupled to a unique catheter.

As shown in FIG. 5B, the lid 810 can include a filter housing 812. The filter housing can be disposed inside the reservoir 850 when the lid 810 is secured to the reservoir 850. When secured to the reservoir 850, the filter housing 812 can be positioned on a top portion 850a of the reservoir 850. The filter housing 812 can receive a filter (not shown). The filter can be removably secured to the filter housing 812. The filter can prevent blood overflow and thus prevent the blood flowing out of the reservoir 850. For example, the filter can absorb blood as the blood collects inside the reservoir 850 and reaches the top portion 850a of the reservoir 850. The filter can also provide a visual indication to clinicians about the amount of blood inside the reservoir 850. For instance, the filter may turn red as the filter absorbs blood, which may provide an indication that blood has reached the top portion 850a of the reservoir 850.

As shown in FIGS. 5A-5C, the ports 800a, 800b, and 802 can have the same structure as one another, for example, to allow coupling to standardized tubing connections.

In certain embodiments, one of the ports 800a and 800b may be closed or sealed before or during an aspiration procedure. For example, in procedures, aspiration may be provided to only a single catheter during an aspiration procedure. In other procedures, aspiration may be performed through two catheters, each coupled to one of the ports 800a and 800b, and one of catheters may be removed during the procedure. In such procedures, the port 800a or 800b in communication with the removed catheter may be closed or sealed to prevent a loss of vacuum pressure in the canister.

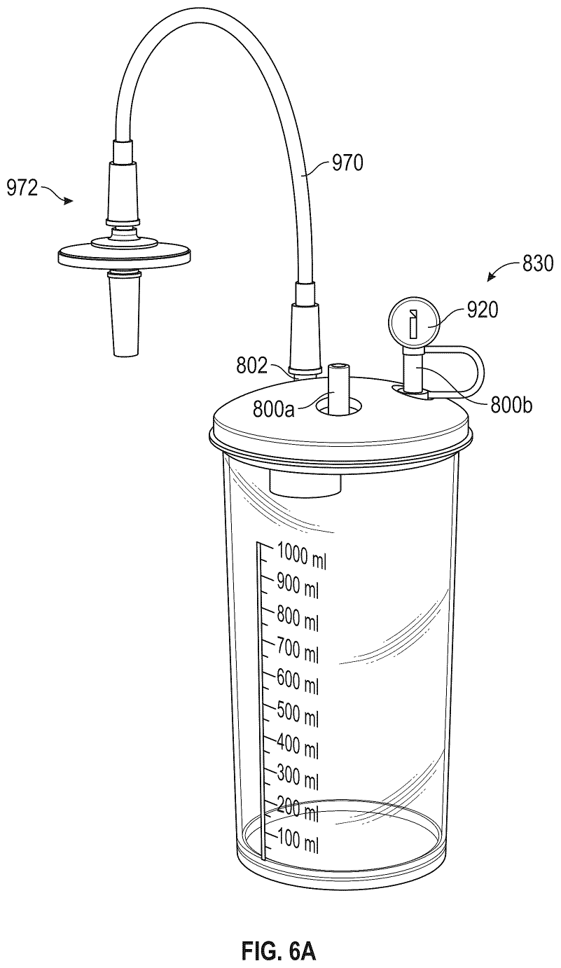

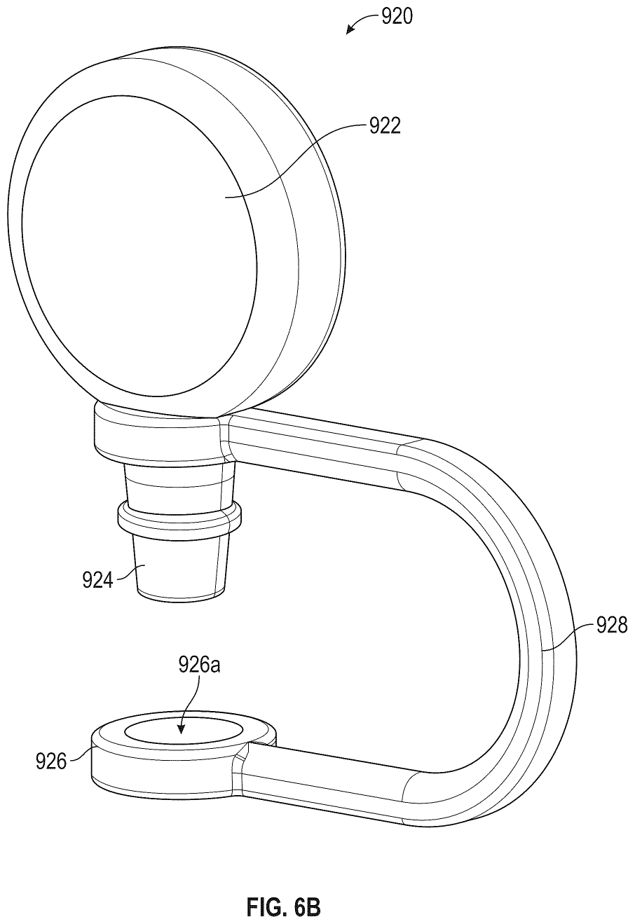

In some embodiments, as shown in FIG. 6A, a cap 920 can be secured to the first port 800a and/or the second port 800b to seal the first and/or second ports 800a, 800b. When only one vacuum line is secured to one of the first and/or second ports 800a, 800b and the other is not use, the cap 920 can be secured to the port not in use. When secured to a port, the cap 920 can beneficially seal the port thus preventing aspiration through that port. This can beneficially increase or maintain the vacuum pressure at the port that is in use by preventing aspiration at the port not in use.

Also shown in FIG. 6A is an embodiment of an aspiration tube 970 and connection 972. The aspiration tube 970 can be connected to the aspiration port 802. The connection 972 can facilitate connection of the aspiration tube 970 to an aspiration pump, such as aspiration pump 260. The aspiration pump can be in fluid communication with the aspiration canister 830 and provide aspiration to the first and second ports 800a, 800b via the aspiration tube 970. In some embodiments, the connection 972 can include an addition filter, which may provide redundancy, to prevent the flow of undesirable material into the aspiration pump.

As shown in FIG. 6B, the cap 920 can include a tab 922, a stopper 924, a ring portion 926, and a connecting member 928. The ring portion 926 can include an opening 926a which can receive a port, such as ports 800a, 800b. The cap 920 can be secured to the to the first and/or second ports 800a, 800b by inserting the first and/or second ports 800a, 800b through the opening 926a of the ring portion 926. The stopper 924 can be inserted into an opening of the first and/or second ports 800a, 800b. Contact between the stopper 924 and an inner surface of the first and/or second ports 800a, 800b can beneficially create a seal. This can beneficially stop aspiration at the first and/or second ports 800a, 800b. The cap 920 can include a flexible and/or stretchable material. The stopper 924 can be removed from the first and/or second ports 800a, 800b by pulling the tab 922. This can beneficially allow clinicians to easily seal and/or open the ports 800a, 800b and/or connect additional vacuum lines to the ports 800a, 800b as needed.

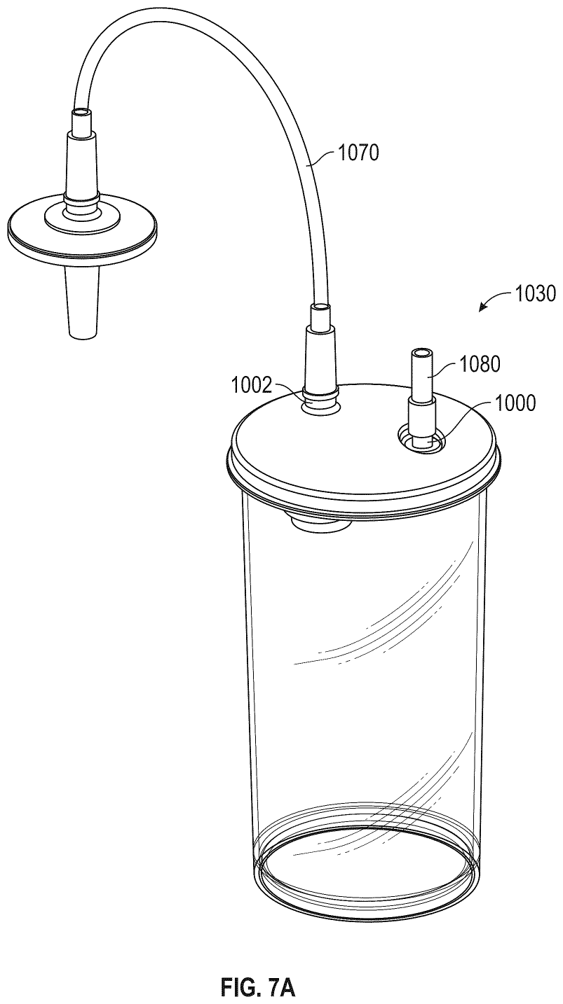

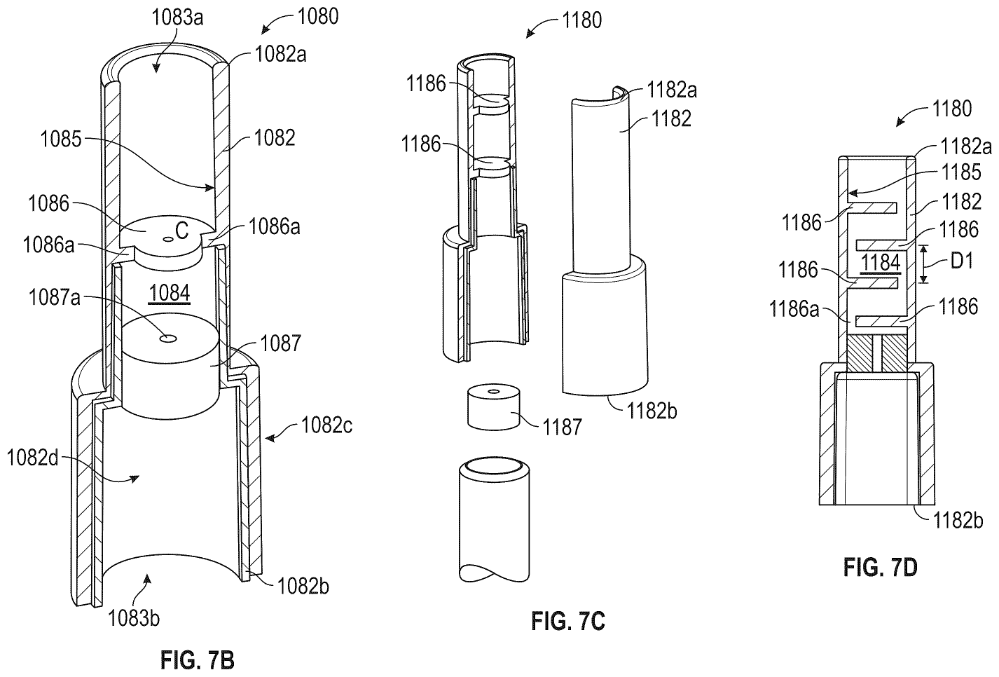

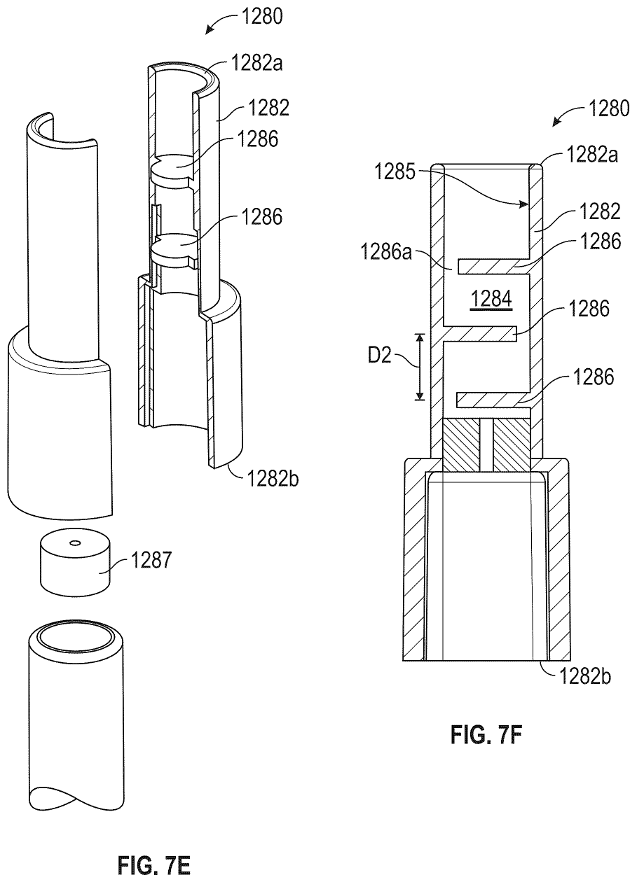

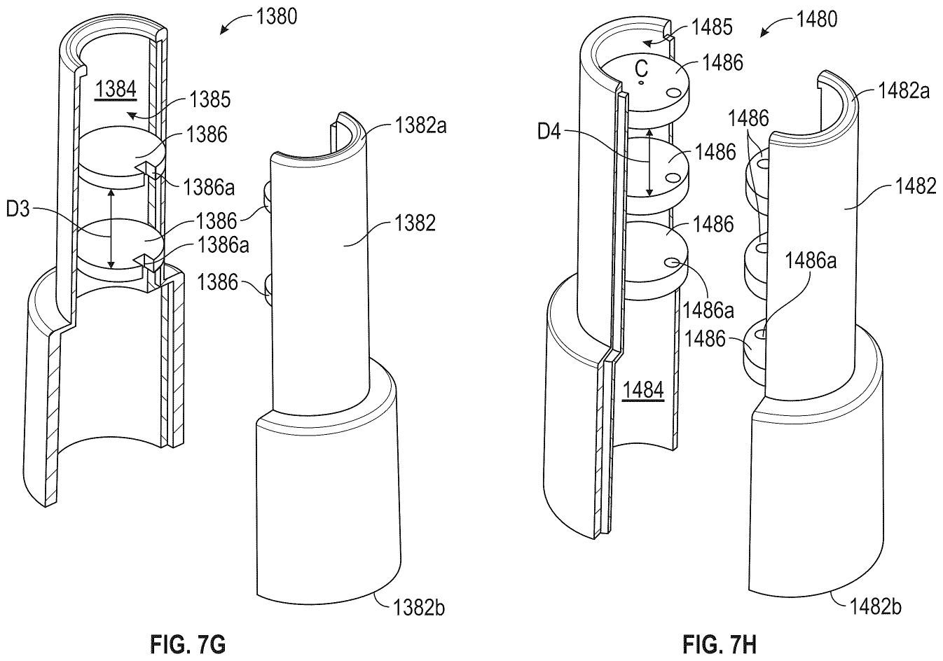

FIG. 7A shows an embodiment of an aspiration canister with a single port for coupling the canister with a catheter, an aspiration port for coupling the catheter with a vacuum source, and a flow reducer secured to the port. The aspiration canister 1030 can include a port 1000 for coupling the canister with a catheter and an aspiration port 1002 for coupling the canister with a vacuum source. Although the aspiration canister 1030 shown in FIG. 7A includes a single port 1000, the aspiration canister can include more than one port 1000 (e.g., two, three, etc.) in some embodiments. A flow reducer 1080 can be secured to the port 1000. As will be further described, the flow reducer 1080 can be configured to reduce the flow rate through the flow reducer 1080 and into the canister 1030. This can beneficially reduce blood loss during a procedure (e.g., a thrombectomy procedure). In certain embodiments, the flow reducer 1080 can be configured to reduce the flow rate while maintaining sufficient vacuum to aspirate a clot or prevent dislodgement of a clot engaged with or corked on a distal end of a catheter.

FIG. 7B shows a cross section of an embodiment of a flow reducer 1080. The flow reducer 1080 can include a main body 1082 having a first end 1082a and a second end 1082b. The first end 1082a can include a first opening 1083a and the second end 1082b can include a second opening 1083b. A fluid flow path 1084 can extend between the first opening 1083a and the second opening 1083b. The flow reducer 1080 can be secured to a port 1000 by positioning the flow reducer 1080 over the port 1000 so that the second opening 1083b receives the port 1000. In some embodiments, a lower portion 1082c of the main body 1082 can include a cavity 1082d. A shape of the cavity 1082d can correspond to a shape of the port 1000. This can beneficially allow the cavity 1082d to receive and secure the port 1000. A friction fit between the cavity 1082d and the flow reducer 1080 can prevent the flow reducer 1080 from accidentally being dislodged from the port 1000.

In some embodiments, one or more baffles can be positioned along the fluid flow path 1084. For example, and as shown in FIG. 7B, the main body 1082 of the flow reducer 1080 can include a baffle 1086 extending from an inner surface 1085 of the main body 1082. To allow aspiration and/or flow of blood through the fluid flow path 1084, the baffle 1086 can include one or more slots 1086a. In some embodiments, the one or more slots 1086a can be positioned along an edge of the baffle 1086 so that the one or more slots 1086a are adjacent the inner surface 1085 of the main body 1082, as shown in FIG. 7B. The one or more slots 1086a can be positioned closer to a center point C of the baffle so that the one or more slots 1086a are not adjacent the inner surface 1085 of the main body 1082. The flow reducer 1080 can include a restrictor 1087. The restrictor 1087 can include a hole 1087a which can extend across the entire restrictor 1087. In some embodiments, the hole 1087a has a diameter of about 1 mm, 2 mm, 3 mm, 4 mm, 5 mm, or any other suitable diameter. As will be further described, when aspiration is applied to the canister 1030 via the aspiration tube 1070, the restrictor 1087 and baffle 1086 can reduce fluid flow rate along the fluid flow path 1084. Although reference is made to the flow reducer 1080 including one baffle 1086, the flow reducer can include more than one baffle 1086 (e.g., two, three, four, five, six, etc.) Additional baffles may further reduce the fluid flow rate along the flow path 1084. For example, the main body 1082 can include a second baffle (not shown) extending from the inner surface 1085 of the main body 1082.

FIGS. 7C-7D show an embodiment of a flow reducer 1180 including four baffles 1186. The flow reducer 1180 can include any of the same or similar features and functions as the flow reducer 1080, which is described in relation to FIGS. 7A-7B. Each of the baffles 1186 can include a slot 1186a. Each slot 1186a can be positioned along an edge of each baffle 1186 so that each slot 1186a is adjacent an inner surface 1185 of the main body 1182. In some embodiments, the slots 1186a from directly adjacent baffles 1186 can be positioned on opposite regions along the perimeter of their respective baffles 1186 so as to increase or maximize the distance between the slots 1186a. This can beneficially increase a length of the fluid flow path 1184, and consequently decrease the flow rate along the fluid flow path 1184 extending between the first end 1182a and the second end 1182b of the main body 1182. In some embodiments, the baffles 1186 can be vertically spaced from each other by a distance D1. In some embodiments, the distance D1 can be about 1 mm. In some embodiments, the distance D1 can be about 0.5 mm, about 1.5 mm, or any other suitable distance. In some embodiments, the flow reducer 1180 with four baffles 1186 and a restrictor 1187 may reduce flow from an aspiration catheter connected to an aspiration cannister, such as aspiration cannister 1030, by about 35%, at least about 35%, or up to about 35%. In some embodiments, the flow reducer 1180 may reduce flow by between 25% and 45% or any other suitable amount.

FIG. 7E-7F show an embodiment of a flow reducer 1280 including three baffles 1286. The flow reducer 1280 can include any of the same or similar features and functions as the flow reducers 1080, 1180, which are described in relation to FIGS. 7A-7D. Each of the baffles 1286 can include a slot 1286a. Each slot 1286a can be positioned along an edge of each baffle 1286 so that each slot 1286a is adjacent an inner surface 1285 of the main body 1282. In some embodiments, the slots 1286a from directly adjacent baffles 1286 can be positioned on opposite regions along the perimeter of their respective baffles 1186 so as to increase or maximize the distance between the slots 1286a. This can beneficially increase a length of the fluid flow path 1284, and consequently decrease the flow rate along the fluid flow path 1284 extending between the first end 1282a and the second end 1282b of the main body 1282. In some embodiments, the baffles 1286 can be vertically spaced from each other by a distance D2. In some embodiments, the distance D2 can be about 1 mm. In some embodiments, the distance D2 can be about 0.5 mm, about 1.5 mm, or any other suitable distance. In some embodiments, the flow reducer 1280 with three baffles 1286 and a restrictor 1287 may reduce flow from an aspiration catheter connected to an aspiration cannister, such as aspiration cannister 1030, by about 27%, at least about 27%, or up to about 27%. In some embodiments, the flow reducer 1280 may reduce flow by between 17% and 35% or any other suitable amount. In certain embodiments, a flow reducer 1280 may reduce flow by an amount less than a flow reducer 1180 under the same conditions (e.g., to due the great number of baffles and/or longer flow path of the flow reducer 1180).

FIG. 7G shows an embodiment of a flow reducer 1380 including four baffles 1386. Each baffle 1386 may have a thickness of about 1 mm. In some embodiments, the thickness can be about 0.5 mm, about 1.5 mm, or any other suitable thickness. The flow reducer 1380 can include any of the same or similar features and functions as the flow reducers 1080, 1180, 1280 which are described in relation to FIGS. 7A-7F. Each of the baffles 1386 can include a slot 1386a. Each slot 1386a can be positioned along an edge of each baffle 1386 so that each slot 1386a is adjacent an inner surface 1385 of the main body 1382. Each slot 1386a can be a substantially square shape. For instance, the slots 1386a can measure about 1 mm by 1 mm.

In some embodiments, the slots 1386a from directly adjacent baffles 1386 can be positioned on opposite regions along the perimeter of their respective baffles 1386 so as to increase or maximize the distance between the slots 1386a. This can beneficially increase a length of the fluid flow path 1384, and consequently decrease the flow rate along the fluid flow path 1384 extending between the first end 1382a and the second end 1382b of the main body 1382. In some embodiments, the baffles 1386 can be vertically spaced from each other by a distance D3. In some embodiments, the distance D3 can be about 1 mm. In some embodiments, the distance D3 can be about 0.5 mm, about 1.5 mm, or any other suitable distance. In some embodiments, the flow reducer 1380 with four baffles 1386 and a restrictor (not shown) may reduce flow from an aspiration catheter connected to an aspiration cannister, such as aspiration cannister 1030, by about 31%, at least about 31%, or up to about 31%. In some embodiments, the flow reducer 1380 may reduce flow by between 21% and 41% or any other suitable amount. In certain embodiments, a flow reducer 1280 may reduce flow by an amount less than a flow reducer 1180 under the same conditions (e.g., to due the great number of baffles and/or longer flow path of the flow reducer 1180).

FIG. 7H shows an embodiment of a flow reducer 1480 including six baffles 1486. Each baffle 1486 may have a thickness of about 1.5 mm. The flow reducer 1480 can include any of the same or similar features and functions as the flow reducers 1080, 1180, 1280, 1380 which are described in relation to FIGS. 7A-7G. Each of the baffles 1486 can include a slot 1486a. Each slot 1486a can be positioned between an edge of the baffles 1486 and a center point C of the baffle 1486 so that the slots 1486a are not directly adjacent to the inner surface 1485 of the main body 1482. Each slot 1486a can include a substantially circular shape.

In some embodiments, the slots 1486a from directly adjacent baffles 1486 can be positioned on opposite regions around the center point C of their respective baffles 1486 so as to increase or maximize the distance between the slots 1486a. This can beneficially increase a length of the fluid flow path 1484, and consequently decrease the flow rate along the fluid flow path 1484 extending between the first end 1482a and the second end 1482b of the main body 1482. In some embodiments, the baffles 1486 can be vertically spaced from each other by a distance D4. In some embodiments, the distance D4 can be about 1 mm. In some embodiments, the flow reducer 1480 with six baffles 1486 and a restrictor (not shown) may reduce flow from an aspiration catheter connected to an aspiration cannister, such as aspiration cannister 1030, by about 46%, at least about 46%, or up to about 46%. In some embodiments, the flow reducer 1480 may reduce flow by between 36% and 56% or any other suitable amount.

While shown and described with respect to the aspiration canister 1030 and port 1000, one of skill in the art would understand that the flow reducer 1080 and other flow reducers described herein can be used with or modified to be used with any of the canisters described herein to reduce the flow rate of fluid into the aspiration canister. As described in further detail herein, in certain embodiments, a flow reducer may be used in combination with an aspiration canister having two ports for coupling to two catheters. For example, the flow reducer can be placed on one of the ports to reduce the flow rate from one of the catheters. Alternatively, a flow reducer may be placed on each of the ports to reduce the flow rate from each catheter.

Although the flow reducers described herein are described and shown as being secured to a port of the aspiration canister, the flow reducer can be positioned distal to the port(s) of the aspiration canister in some embodiments. For example, a flow reducer 1580 can be positioned at or near a distal end 1590b of a vacuum or aspiration line 1590, as shown in FIG. 7I. The aspiration line 1590 can be connected to a port 1500 by securing a proximal end 1590a of the aspiration line 1590 to a port 1500 of a canister 1530. The distal end 1590b of the aspiration line 1590 can be coupled to an aspiration catheter 1571, This can beneficially place the aspiration line 1590 in fluid communication with the aspiration canister 1530 and thus provide aspiration to the aspiration line 1590 and the catheter 1571.

The flow reducer 1580 can reduce the flow rate of fluid through the flow reducer 1580 and proximal of the flow reducer 1580. Flow rate distal of the flow reducer 1580 may be greater than the flow rate proximal of the reducer 1580. Accordingly, the flow reducer 1580 may desirably be positioned distally along the aspiration line 1590 to provide a greater length of aspiration line 1590 or flow path having a reduced flow rate and a shorter length of the aspiration line or flow path without a reduced flow rate. This may beneficially reduce a total amount of blood aspirated into the aspiration canister 1530 over a particular period of time in comparison to a flow reducer positioned more proximally along the aspiration line 1590 and/or directly connected to a port of the aspiration canister 1530.

The aspiration canister 1530 can have any of the same or similar features and functions as any of the other aspiration canisters described herein.

As shown in FIG. 7I, the aspiration line 1590 can include a clot pod or clot retrieval device 1572 for retaining an aspirated clot therein. The aspiration catheter 1571 can be advanced though the vasculature of a patient so that aspiration at a distal end 1571b of the aspiration catheter 1571 causes the aspiration catheter 1571 to aspirate clots from the vasculature of a patient into the clot retrieval device 1572. The clot retrieval device 1572 can beneficially allow for visualization of the clot once the clot is removed from the vasculature of the patient. Various examples of clot retrieval devices are shown and described in International Patent Application No. PCT/US2022/078113, filed Oct. 14, 2022, the contents of which are herein incorporated by reference in their entirety.

As shown in FIG. 7I, the flow reducer 1580 may be positioned along the aspiration line 1590 proximal to the clot retrieval device 1572 to prevent or restrict a clot from entering the flow reducer 1580. In some embodiments, the flow reducer 1580 may be directly connected to a proximal end of the clot retrieval device 1572 to increase the length of the flow path having a reduced flow rate.

Although reference is made to the aspiration canister 1530 including a single port 1500, the aspiration canister 1530 can include more than one port with an aspiration line, a flow reducer, and/or a catheter in fluid communication with the aspiration canister 1530. Additionally, the aspiration line 1590 may be used with any of the aspiration canisters described herein.

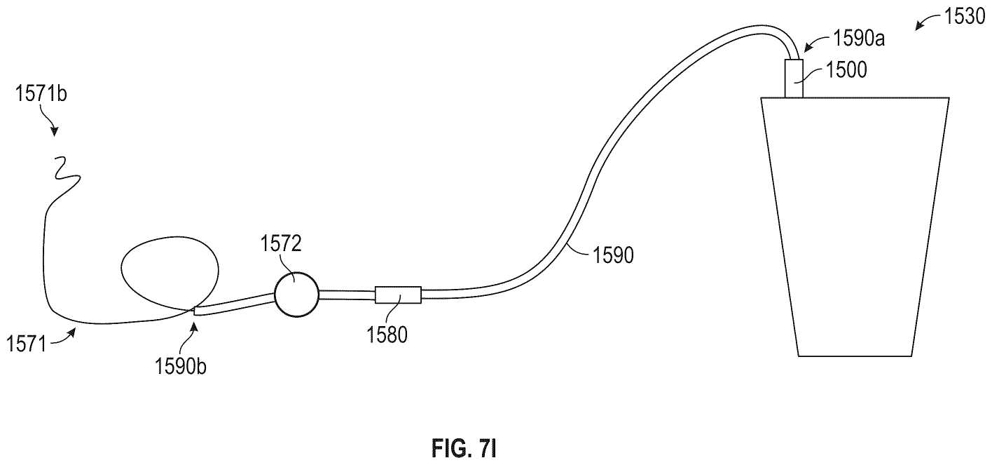

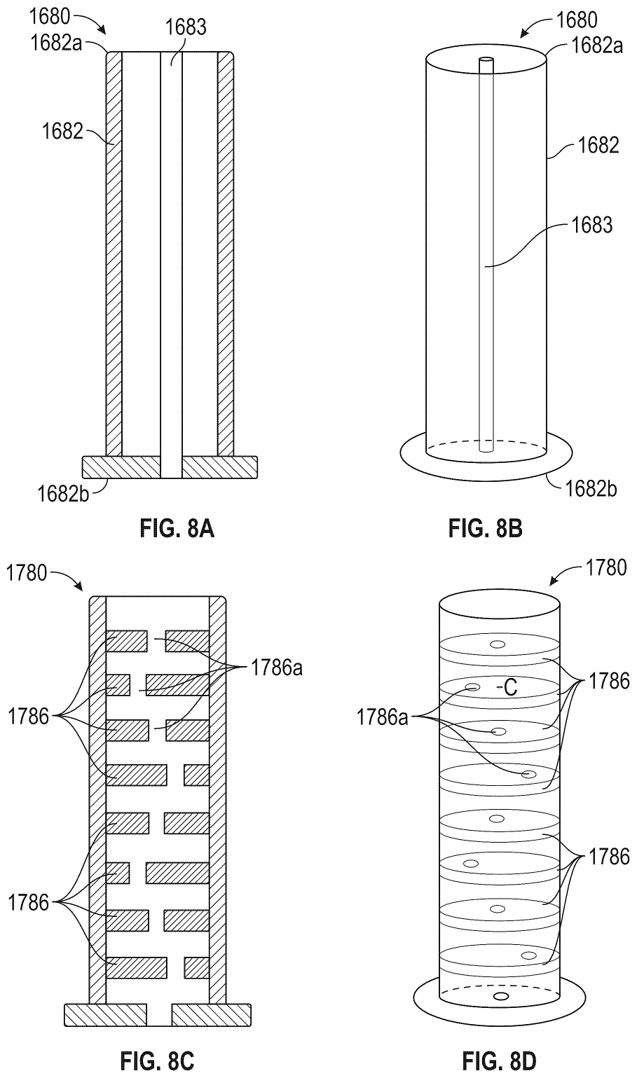

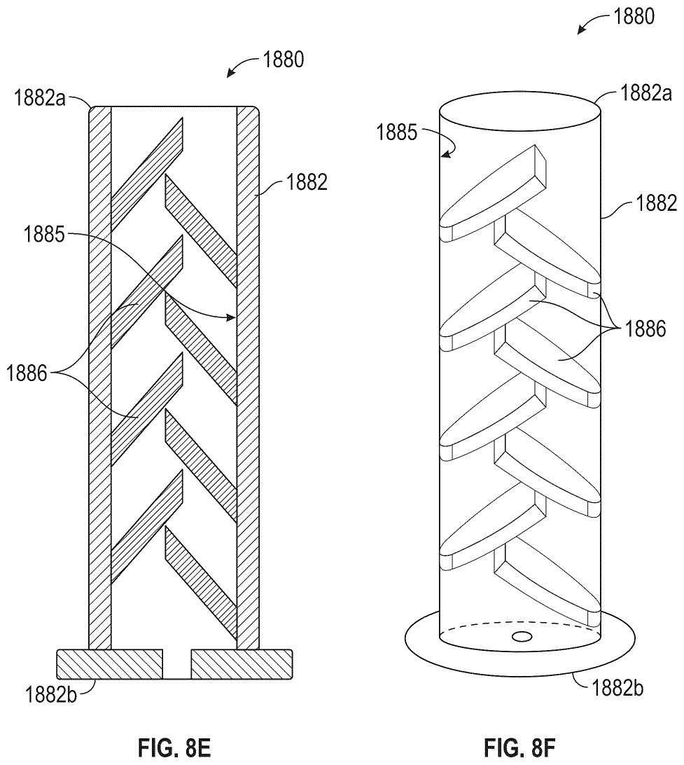

FIGS. 8A-8F show additional embodiments of flow reducers for use with an aspiration canister. As shown in FIGS. 8A-8B, a flow reducer 1680 can include a single opening 1683 extending from a first end 1682a to a second end 1682b of a main body 1682 of the flow reducer 1680. A flow reducer 1780, as shown in FIGS. 8C-8D, can include eight baffles 1786. Each baffle 1786 can include a slot 1786a. The baffles 1786 may include one baffle 1786 with a slot 1786a positioned substantially along a center point of the baffle 1786. Two baffles 1786 separated by a baffle 1786 having a slot 1786a positioned substantially along a center point of the baffle 1786, can include slots 1786a positioned on opposite regions around the center point C of their respective baffles 1786. The slots 1786a can include a substantially circular shape. In some embodiments, the flow reducer can include baffles extending from an inner surface of the flow reducer at an angle other than 90°. For example, as shown in FIGS. 8E-8F, a flow reducer 1880 can include eight baffles 1886. Directly adjacent baffles 1886 can extend from opposite sides of an inner surface 1885 of the main body 1882. A gap G between each pair of baffles 1886 allows fluid flow between a first end 1882a and a second end 1882b of the main body 1882 while reducing flow rate.

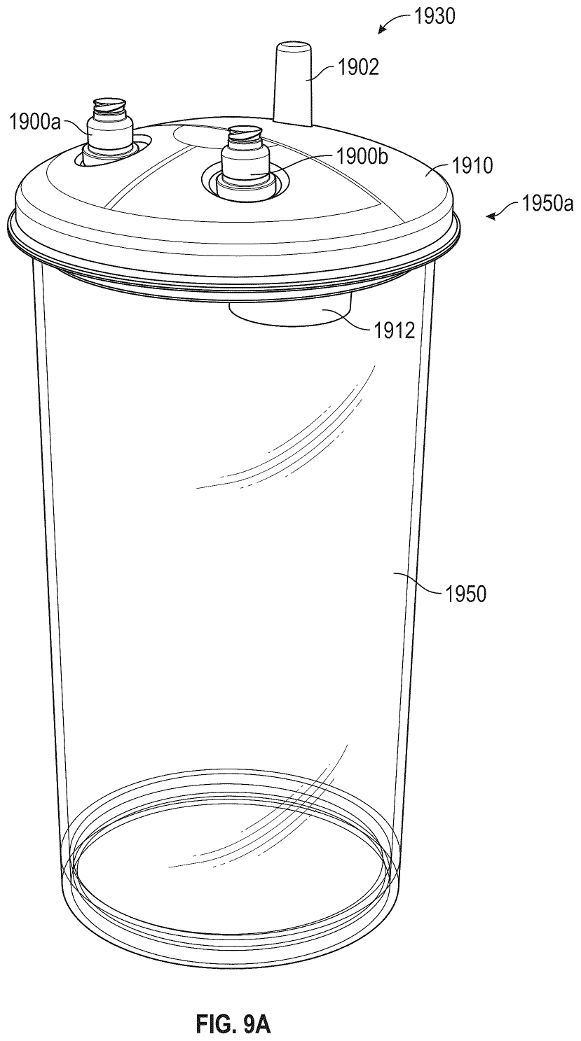

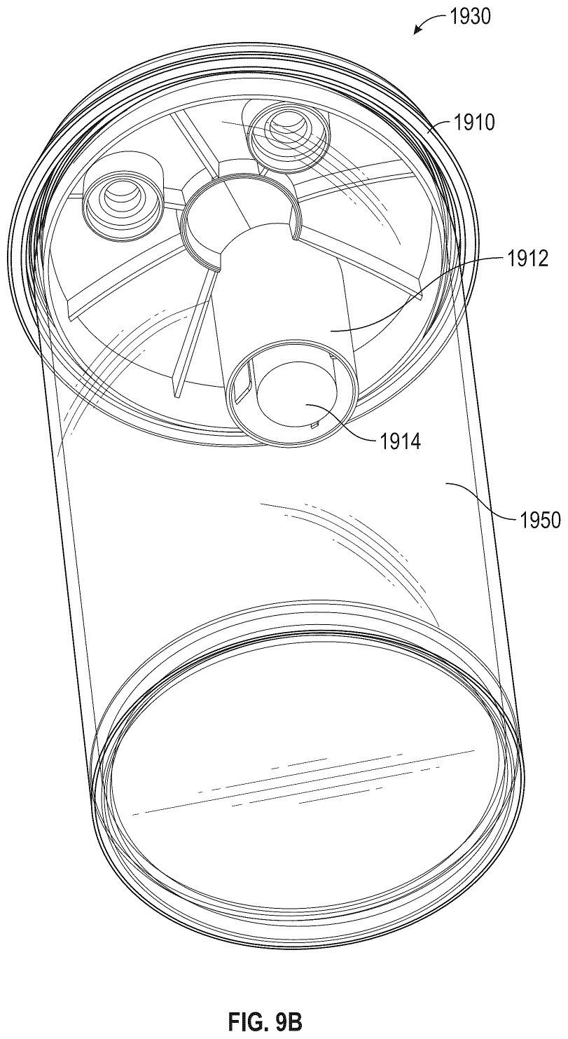

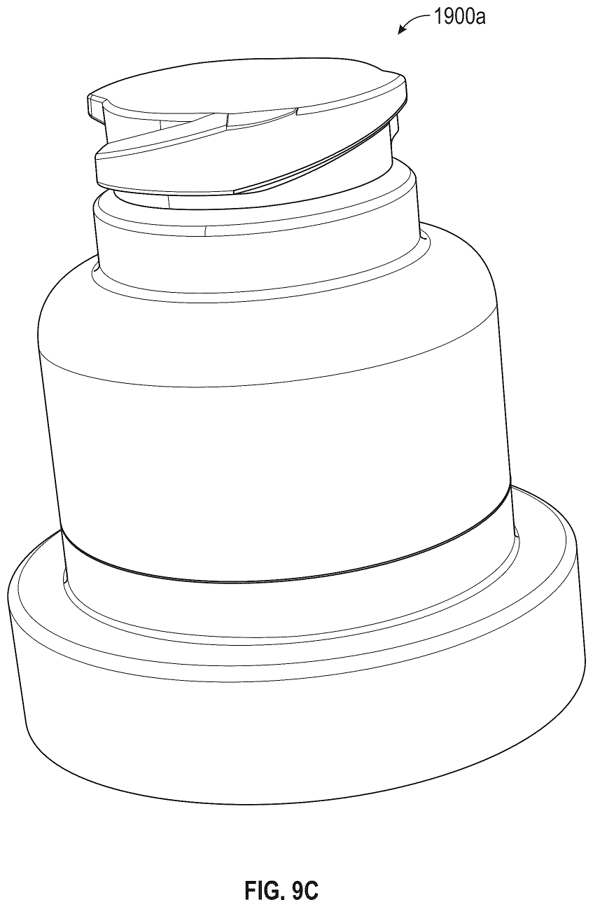

FIGS. 9A-9B show an embodiment of an aspiration canister with self-shutting or self-sealing ports. The aspiration canister 1930 can include any of the same or similar features and functions as the other aspiration canisters described herein. The aspiration canister 1930 can include a first self-sealing port 1900a, a second self-sealing port 1900b, and an aspiration port 1902. A vacuum line (not shown), which can include any of the same or similar features and functions as the vacuum line 740 or vacuum line 1590, can be secured to each of the first and second self-sealing ports 1900a, 1900b.

As shown in FIGS. 9A-9B, the ports 1900a and 1900b may have a different structure than the aspiration port 1902 that couples the canister 1930 to the aspiration pump. This may prevent accidental coupling of one of the vacuum lines connected to a catheter directly to the aspiration pump.

As shown in FIG. 9B, a lid 1910 of the aspiration canister 1930 can include a filter housing 1912. The filter housing can be disposed inside the reservoir 1950 when the lid 1910 is secured to the reservoir 1950. When secured to the reservoir 1950, the filter housing 1912 can be positioned on a top portion 1950a of the reservoir 1950. The filter housing 1912 can receive a filter 1914. The filter 1914 can be removably secured to the filter housing 1912. The filter 1914 can prevent blood overflow and thus prevent the blood flowing out of the reservoir 1950. For example, the filter 1914 can absorb blood as the blood collects inside the reservoir 1950 and reaches the top portion 1950a of the reservoir 1950. As discussed in relation to FIGS. 5A-5C, the filter 1914 can also provide a visual indication about the amount of blood inside the reservoir 1950. For instance, the filter 1914 may turn red as the filter 1914 absorbs blood which may provide an indication that blood has reached the top portion 1950a of the reservoir 1950.

FIG. 9C shows an embodiment of a perspective view of a self-sealing port 1900a. While a port 1900a is shown, one of skill in the art would understand that the port 1900b can include any of the same or similar features and functions. The self-sealing port 1900a enables a vacuum line to be added and removed as needed. The self-sealing port 1900a can be self-shutting or self-sealing, such that when the port is not connected to a vacuum line, the self-sealing port 1900a is closed and vacuum pressure is substantially maintained, or vacuum pressure is not substantially lost within the aspiration canister. The self-sealing port 1900a can beneficially allow the connection of a second aspiration line to an aspiration canister without significant vacuum pressure loss and/or allow immediate vacuum pressure at the second aspiration line. This can prevent vacuum pressure loss along a first catheter connected to a first aspiration line when a second catheter is disconnected from the aspiration canister.

In some instances, the self-sealing ports 1900a and 1900b allow a physician to add additional aspiration lines at any stage during a procedure. Any additional aspiration lines (e.g., a secondary line) may be fluidly connected to one of the ports 1900a and 1900b at any stage prior to or during an aspiration procedure. In some instances, multiple aspiration lines may be added and/or removed during an aspiration procedure. For example, during an aspiration procedure, a healthcare practitioner may desire to include an additional aspiration line to provide additional aspiration through a secondary device as described herein (e.g., a secondary catheter or a guide catheter) by connecting a new aspiration line between one of the self-sealing ports and the secondary device during the procedure. Aspiration through the secondary device may occur as an alternative or in addition to aspiration through a primary device (e.g., a first catheter). In contrast, ports that are not self-sealing may result in an unacceptable loss of pressure when a line is not connected to the port, preventing replacement or addition of an aspiration line during an aspiration procedure. The ports 1900a and 1900b may comprise any suitable closeable needless valve connector.

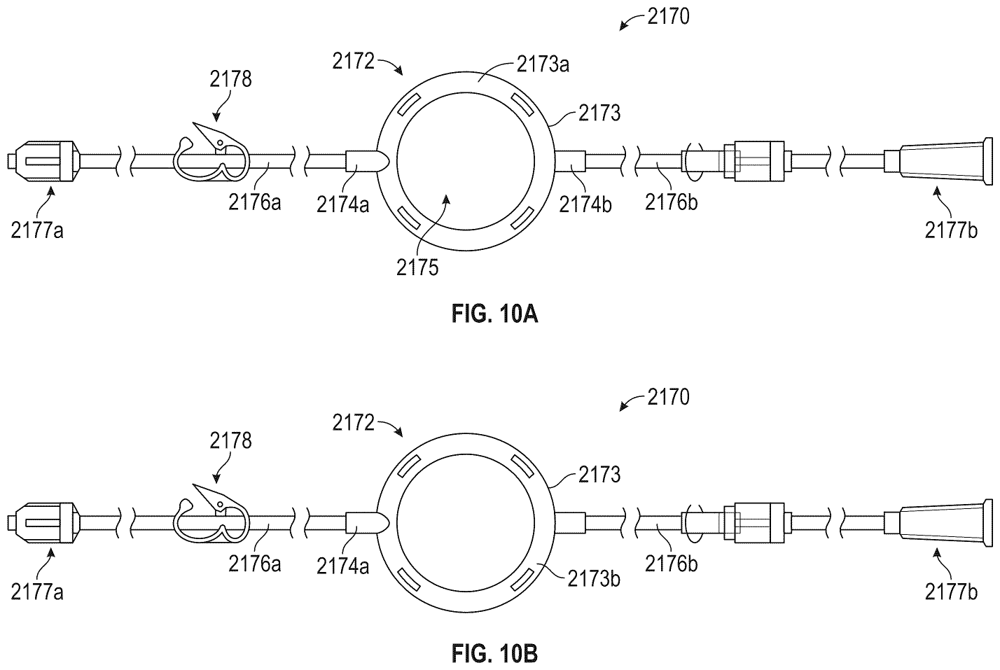

FIGS. 10A-10B illustrate an embodiment of an aspiration line 2170 including a clot retrieval device 2172. The aspiration line 2170 can include any of the same or similar features and functions as the aspiration line 1590, which is described in relation to FIG. 7I. The clot retrieval device 2172 can include a body 2173 enclosing a chamber which communicates with a first port 2174a and a second port 2174b. In some embodiments, the body 2173 can include a flush port (not illustrated) that is configured to allow the injection of saline or other fluid into the chamber to improve clot visualization once it is trapped in a filter 2175.

In some embodiments, the body 2173 includes a housing having a top portion 2173a and a bottom portion 2173b. In some examples, the body 2173 includes a filter 2175 positioned in the chamber between the top portion 2173a and the bottom portion 2173b. In some examples, the first port 2174a is configured to connect to a first end of a tubing section 2176a that is fluidly connected to a proximal end of an aspiration catheter, such as aspiration catheter 1571 (e.g., to a valve, such as an RHV, of the catheter). In some embodiments, the first tubing section 2176a includes a connector 2177a positioned at a second end of the first tubing section 2176a that is configured to engage or mate with a corresponding connector of the aspiration catheter (e.g., of a valve, such as an RHV, of the catheter).

In some embodiments the second port 2174b is configured to connect to a first end of a second tubing section 2176b that is fluidly connected to an aspiration canister, such as aspiration canister 1530, in communication with an aspiration source (e.g., a pump). In some embodiments, the second tubing section 2176b includes a connector 2177b positioned at a second end of the second tubing section 2176b that is configured to engage or mate with a corresponding connector of the aspiration canister, and/or flow reducer coupled to the port of the canister. For example, the connector 2177b can be connected to a port, which can include any of the same or similar features and functions as ports 800a, 800b, and/or a flow reducer, which can include any of the same or similar features and functions as the flow reducer 1080 or any other flow reducers described herein. In some embodiments, the aspiration line 2170 can include a clamp 2178. The clamp 2178 can be positioned over the first tubing section 2176a to allow the user to engage the clamp 2178 and control fluid flow along the aspiration line 2170. For example, the clamp 2178 can be closed to restrict and/or reduce flow along the aspiration line 2170 proximal to the clamp 2178 and opened to allow and/or increase the fluid flow along the aspiration line 2170 proximal to the clamp 2178. In some embodiments, at least a portion of the body 2173 of the clot retrieval device 2172 can be optically transparent to improve clot visualization once the clot is trapped in the clot retrieval device 2172.

In some embodiments, the filter 2175 can be circular in shape. In some embodiments, the filter 2175 can be secured between the top portion 2173a and the bottom portion 2173b of the body 2173. In some examples, the filter 2175 of the clot retrieval device 2172 may be provided with different pore sizes based on the needs of the physician or the patient. For example, in some embodiments, the filter 2175 can include 1 mm holes that catches solid clot debris as blood is driven through the aspiration line 2170. In some embodiments, the pore sizes may be at most 1 mm, at most 2 mm, at most 3 mm, at most 4 mm, at most 5 mm, etc.

In some embodiments, the aspiration line 2170 aspirates blood which can flow through the first tubing section 2176a and enter the clot retrieval device 2172 of the aspiration line 2170. The aspirated blood can then be filtered by the filter 2175. Material that is greater in size than the openings on the filter 2175 is prevented from leaving the clot retrieval device 2172, while material that is smaller in size than the openings on the filter 2175 flows out of the clot retrieval device 2172 via the second tubing section 2176b. In some embodiments, the user can slow down fluid flow along the aspiration line 2170 by engaging the clamp 2178. In some embodiments, the user can inspect the clot or other larger aspirated material caught by the clot retrieval device 2172. In some embodiments, the material trapped in the clot retrieval device 2172 can be removed from the clot retrieval device 2172 and tested by the user.

The first port 2174a may be larger, smaller, or the same size as the second port 2174b and/or may be configured to fluidly connect tubing sections that are larger, smaller, or the same size as the second port 2174b. In some instances, the first port 2174a may be larger than the second port 2174b such that the first port 2174a is configured to fluidly connect to a first tube or tubing section (e.g., first tubing section 2176a) being larger than a second tube or tubing section (e.g., second tubing section 2176b) that may be fluidly connected to the second port 2174b. The first port 2174a, in some embodiments, may be configured to fluidly connect to a standard tube (e.g., a tube having an inner diameter of about 0.1 inches). The second port 2174b may be smaller than the first port 2174a such that second tubing section 2176b is smaller than first tubing section 2176a. In some instances, the smaller size of the second tubing section 2176b may advantageously facilitate in decreasing a priming time of the system such that the aspiration source is configured to “prime” (e.g., apply negative pressure at least throughout the length of the second tubing section 2176b and the clot retrieval device 2172). The smaller tube size may facilitate priming by decreasing the overall internal tube volume between the aspiration source and the clot retrieval device 2172 that requires priming. In some instances, the smaller tube size may advantageously decrease a blood fluid flow rate through the second tubing section 2176b so as to reduce a potential amount of blood loss during an aspiration procedure.

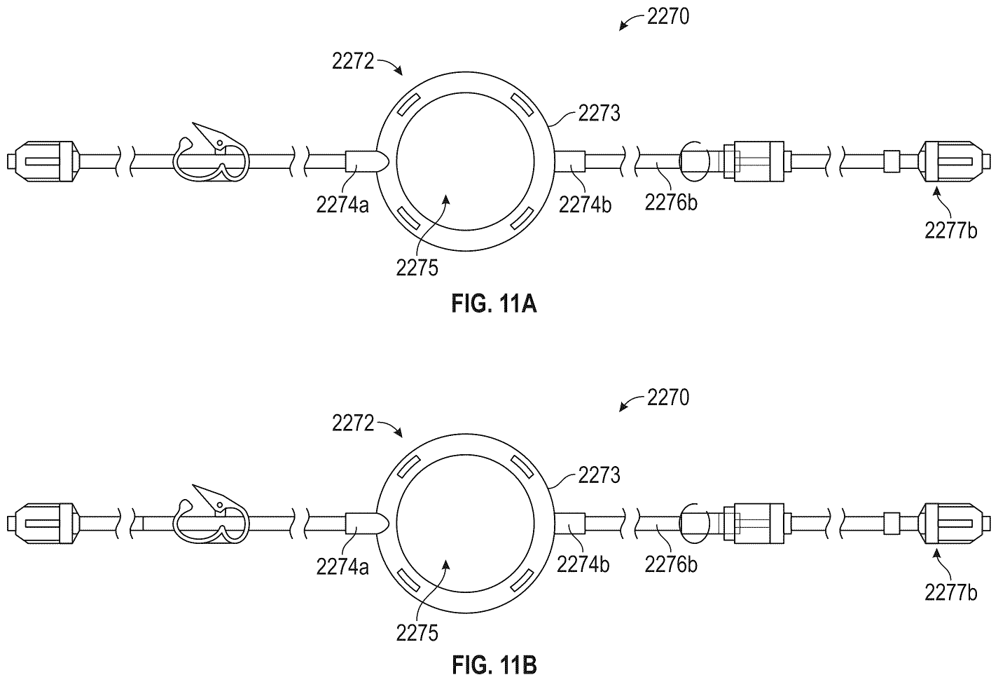

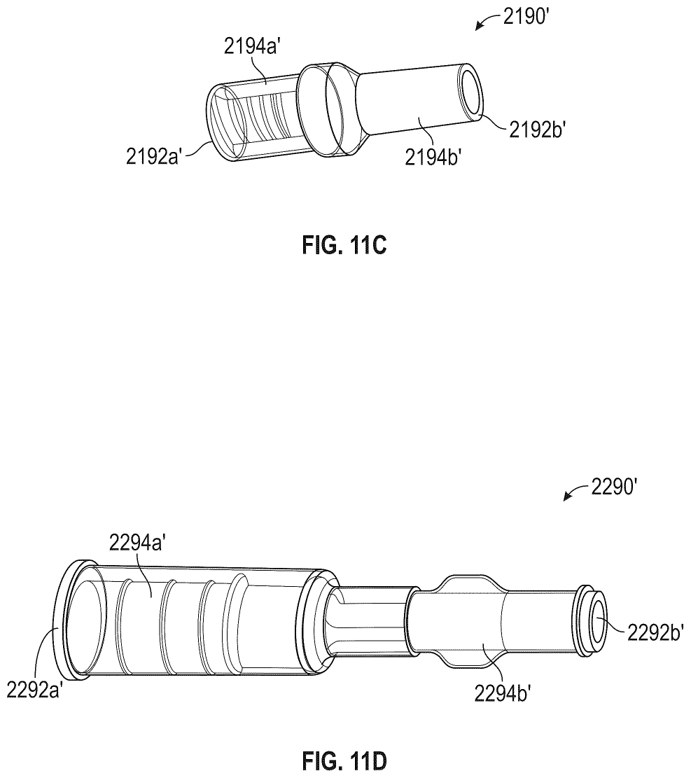

FIGS. 11A-11B illustrate an embodiment of an aspiration line 2270 including a clot retrieval device 2272. The aspiration line 2270 can include any of the same or similar features and functions as the aspiration line 2170, which is described in relation to FIGS. 10A-10B. The clot retrieval device 2272 can include a body 2273 enclosing a chamber which communicates with a first port 2274a and a second port 2274b. In some embodiments, the body 2273 can include a flush port (not illustrated) that is configured to allow the injection of saline or other fluid into the chamber to improve clot visualization once it is trapped in a filter 2275.

In some embodiments, a second port 2274b is configured to connect to a first end of a second tubing section 2276b that is fluidly connected to an aspiration source (e.g., a pump), such as aspiration canister 1930. In some embodiments, the second tubing section 2276b includes a connector 2277b positioned at a second end of the second tubing section 2276b that is configured to engage or mate with a corresponding connector, port, and/or flow reducer coupled to an aspiration container. For example, the connector 2277b can be connected to a port, which can include any of the same or similar features and functions as a self-sealing port, such as self-sealing ports 1900a, 1900b, which are described in relation to FIG. 9A.

FIGS. 11C-11D illustrate embodiments of adapters which can be used with the aspiration lines 2170, 2270. As shown in FIG. 11C, an adapter 2190′ can include a first end 2192a′, a second end 2192b′, a self-sealing port 2194a′ and a coupling member 2194b′. In some embodiments, the adapter 2190′ can be connected to a connector, such as connector 2177b. The adapter 2190b′ can be connected to the connector 2177b by inserting the coupling member 2194b′ into the connector 2177b. A friction fit between the connector 2177b and the coupling member 2194b′ can prevent the coupling member 2194b′ from accidentally being dislodged from the connector 2177b. The self-sealing port 2194a′ can be connected to a self-sealing port, such as self-sealing ports 1900a, 1900b of the aspiration canister 1930. The adapter 2190′ can allow the aspiration line 2170 to be connected to an aspiration canister having self-sealing ports. This can beneficially allow users to connect the aspiration line 2170 to aspiration canisters with self-sealing ports, such as aspiration canister 1930, and aspiration canisters with non-self-sealing ports, such as aspiration canister 830.

As shown in FIG. 11D, an adapter 2290′ can include a first end 2292a′, a second end 2292b′, an attachment member 2294a′ and a coupling member 2294b′. In some embodiments, the adapter 2290′ can be connected to a connector, such as connector 2277b. The adapter 2290b′ can be connected to the connector 2277b by inserting the coupling member 2294b′ into the connector 2277b. A friction fit between the connector 2277b and the coupling member 2294b′ can prevent the coupling member 2294b′ from accidentally being dislodged from the connector 2277b. The attachment member 2294a′ can be connected to a port, such as ports 800a, 800b. The adapter 2290′ can allow the aspiration line 2270 to be connected to an aspiration canister having non-self-sealing ports. This can beneficially allow users to connect the aspiration line 2270 to aspiration canisters with non-self-sealing ports, such as aspiration canister 830, and aspiration canisters with self-sealing ports, such as aspiration canister 1930. DUAL PORT ASPIRATION CANISTER OPERATION

Any of the dual port aspiration canisters described herein can be used to connect one or more catheters. For example, a first catheter can be in fluid communication with an aspiration line connected to a first port (e.g., port 800a of aspiration canister 830; port 1900a of the aspiration canister 1930, etc.), and a second catheter can be connected to a second aspiration line connected to the second port (e.g., port 800b of aspiration canister 830; port 1900b of the aspiration canister 1930, etc.). When connected to the aspiration canister through the first and second aspiration lines, aspiration at distal ends of the first and/or second catheters can be used to remove (e.g., aspirate) clots from a vasculature of a patient.

In some embodiments, the first and second catheters can each have a lumen extending a length of the first and second catheters, respectively. One of the first and second catheters can be coaxially disposed in the lumen of the other of the first and second catheters. For example, the second catheter can include an outer diameter smaller than an inner diameter of the first catheter to allow the second catheter to extend through the lumen of the first catheter. In some embodiments, the first catheter can include an inner diameter of about .088″ and an outer diameter of about .110″. The inner and outer diameters of the first catheter can be smaller or greater that .088″ (e.g., .086″ 0.087″, .089″, .090″, etc.) and .110″ (e.g., .108″, .109″, .111″, .112″, etc.) respectively. In some embodiments, the second catheter can include an inner diameter of about .071″ and an outer diameter of about .085″. The inner and outer diameters of the second catheter can be smaller or greater than .071″ (e.g., .069″ 0.070″, .072″, .073″, etc.) and .085″ (e.g., .083″ 0.084″, .086″, .087″, etc.) respectively. In some cases the outer diameter of the second catheter can be between about .053″ to about .085″, and the inner diameter can between about .035″ to about .071″.

The difference in inner/outer diameter between the first and second catheter may allow one of the first and/or second catheters to have a higher flow rate than the other catheter when aspiration is applied (e.g., in the absence of any obstructions, such as the present of the second catheter within the first catheter). For example, the first catheter may include a lumen having an inner diameter larger than that of the second catheter which may result in the first catheter having a fluid flow rate (in the absence of the second catheter within the first catheter) greater than a fluid flow rate within the first catheter (e.g., with vacuum applied from the same dual port canister). To reduce the fluid flow rate from the first catheter into the canister, a flow reducer, such as flow reducers 1080, 1180, 1280, 1380, 1480, 1580, 1680, 1780, 1880 can be secured along the fluid path from the first catheter to the aspiration canister (e.g., secured to the first port (e.g., port 800a, 1900a, etc.) of the canister or along the aspiration line between the first catheter and the aspiration canister). The flow reducer can beneficially reduce the fluid flow rate proximal to the flow reducer (e.g., where the canister is positioned proximal to the flow reducer and the catheter is positioned distal to the flow reducer). In some embodiments, the flow reducer can cause the fluid flow rate proximal to the flow reducer to approximate the fluid flow rate along the second catheter. As will be further described below, this can beneficially reduce blood loss during a thrombectomy procedure. In certain embodiments, each catheter can include a flow reducer. For example, a flow reducer may be positioned along the flow path from the second catheter to the aspiration canister to reduce the fluid flow rate proximal of the flow reducer.

In a thrombectomy procedure, a first catheter (e.g., a guide catheter) and a second catheter (e.g., an aspiration catheter) may be inserted into the vasculature of a patient to retrieve a clot. The first catheter can be advanced through the vasculature of a patient so that the distal tip of the first catheter is positioned adjacent the clot. The second catheter can be advanced through the first catheter, for example, through a valve such as an RHV at a proximal end of the first catheter, to the location of the clot. A distal end of the second catheter can extend beyond the distal end of the first catheter. Aspiration at the distal end of the first can allow the first catheter to aspirate the clot. In some embodiments (e.g., if a catheter is too large for aspiration by the second catheter, the clot may become engaged with or corked on the distal end of the second catheter. In other embodiments, if the clot is too large for aspiration by the second catheter and/or too large to engage with the distal end of the second catheter, the clot may be aspirated by the first catheter. In other embodiments, if the clot is too large for aspiration by the first catheter, the clot may become engaged with or corked on the distal end of the first catheter.

In some embodiments, (e.g., while a clot is lodged at the distal of the second catheter or after aspiration of at least a portion of the clot by the second catheter), the second catheter can be retracted (e.g., to remove an engaged clot from the vasculature of the patient). The RHV at the proximal end of the first catheter may be opened for removal of the second catheter from the proximal end of the first catheter. When the RHV is open and the second catheter is removed from the RHV, the open state of the RHV results in a lower flow rate and a lower pressure through the aspiration line of the first catheter in comparison to when the RHV is in a closed state. When the second catheter has been removed from the body and aspiration is occurring through the aspiration line coupled to the second catheter, the open state of the aspiration line of the second catheter causes a lower pressure and lower flow rate through the aspiration line of the first catheter.

It is desirable that a substantial loss of vacuum pressure does not occur at the distal end of the first catheter. In some instances, a loss of vacuum pressure to a vacuum pressure below a threshold value may result in dislodgement of a clot, thrombus, or other vascular occlusion that has been captured by the first catheter during an aspiration procedure. In some embodiments, a vacuum pressure of at least −14 inHg may prevent the clot, thrombus, or other vascular occlusion that has been captured by the first catheter during an aspiration procedure from dislodging. In some embodiments, a vacuum pressure of at least −380 mmHg may prevent the clot, thrombus, or other vascular occlusion that has been captured by the first catheter during an aspiration procedure from dislodging. As used herein, when the vacuum pressure is described as a negative value, such as −14 inHg, vacuum pressures below a threshold value refer to vacuum pressures having an absolute value below the absolute value of the threshold value, vacuum pressures above or greater than a threshold value are vacuum pressures having an absolute value greater than the absolute value of the threshold value.

Aspiration through both the first and second catheters can beneficially reduce the risk of the clot being dislodged from the first catheter as the second catheter is retracted from the first catheter.

To maintain or increase the vacuum pressure at the distal end of the first catheter when the second catheter is retracted from the first catheter, aspiration through the aspiration line connected to the second catheter can be prevented (e.g., using a clamp, such as clamp 2178). This can cause the vacuum pressure at the first catheter to increase, thereby reducing the risk of a clot being dislodged from the distal end of the first catheter. Closing of the aspiration line connected to the second catheter may also cause an increase in flow rate through the first aspiration line.

Additionally, to maintain or increase the vacuum pressure at the distal end of the first catheter when the second catheter is retracted from the first catheter, the RHV of the first catheter can be closed. This can cause the vacuum pressure at the second catheter to increase, thereby reducing the risk of a clot being dislodged from the distal end of the second catheter. Closing of the RHV of the first catheter may also cause an increase in flow rate through the first aspiration line.

The second aspiration catheter and/or RHV may be at least temporarily left open during a procedure by a physician. Accordingly, it is desirable that vacuum pressure through the first catheter is sufficient to retain a clot, thrombus or other vascular occlusion while both the second aspiration catheter and/or RHV are open.

As described herein, when the aspiration line of the second catheter is closed and the RHV is closed, the flow rate within the first catheter may increase. As described herein, a flow restrictor may reduce the flow rate to reduce blood loss. However, reduction in flow rate by a flow restrictor may reduce the vacuum pressure within the first catheter. The flow restrictors described herein may be configured to reduce the flow rate below a desired maximum threshold value for blood loss in situations where both the RHV and the aspiration line of the second catheter are closed, while allowing sufficient vacuum pressure in the first catheter to prevent dislodgement of a clot, thrombus, or other vascular occlusion at the distal end of the first catheter. For example, the flow restrictor may reduce the flow rate proximal to the aspiration line connected to the first catheter from about 245 ml/min, when a flow restrictor is not used, to at least about 190 ml/min, at least about 180 ml/min, at least about 170 ml/min, at least about 160 ml/min, at least about 150 ml/min, or any other suitable flow rate. In situations where the aspiration catheter and/or RHV are at least temporarily left open during a procedure by a physician, vacuum pressure through the first catheter can be maintained from about −530 mmHg to about −635 mmHg, which can beneficially reduce the risk of the clot dislodging from the first catheter. In some embodiments, vacuum pressure through the first catheter can be at least about −500 mmHg, −520 mmHg, −550 mmHg, −580 mmHg, −600 mmHg, −620 mmHg, and/or −650 mmHg, even when the aspiration catheter and/or RHV are at least temporarily left open.

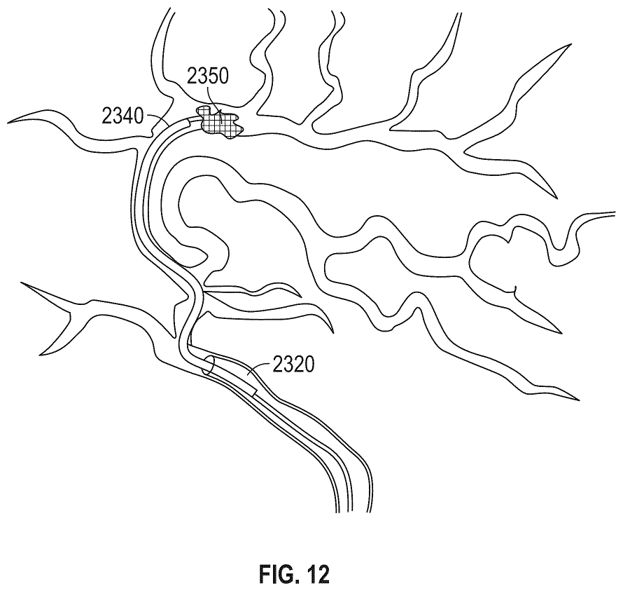

FIG. 12 shows an example of first and second catheters deployed inside the vasculature of a patient. Each of the first aspiration catheter 2320 and the second aspiration catheter 2340 can be in fluid communication with a distinct aspiration line (e.g., aspiration line 1590), a port (e.g., port 1500), and an aspiration canister (e.g., canister 1530). The first catheter 2320 can include a lumen having a diameter larger than an exterior diameter of the second catheter 2340 so as to allow the first catheter 2320 to receive the second catheter 2340 and allow the second catheter 2340 to extend and advance though the lumen of the first catheter 2320.

In some embodiments, a flow reducer (e.g., 1080, 1180, 1280, 1380, 1480, 1580, 1680, 1780, 1880, etc.) can be positioned along the fluid flow path between the catheter 2320 and the canister. In some embodiments, a flow reducer (e.g., 1080, 1180, 1280, 1380, 1480, 1580, 1680, 1780, 1880, etc.) can be positioned along the flow path between the catheter 2340 and the canister.

Fluid connection of the first and second aspiration catheters 2320, 2340 to the aspiration canister can beneficially allow the first and second aspiration catheters 2320, 2340 to apply aspiration when the aspiration catheters 2320, 2340 are connected to the aspiration canister. In some embodiments, the catheter 2320 can be coupled to a valve (e.g., a hemostasis valve or a rotating hemostasis valve), which can be opened to allow for advancement/retraction of the catheter 2340 through the catheter 2320. In some embodiments, the catheter 2340 may also be coupled to a valve (e.g., a hemostasis valve or a rotating hemostasis valve) to allow for advancement of another device (e.g., another catheter) through the catheter 2320

During a thrombectomy procedure, the first catheter 2320 can be advanced through the vasculature of a patient towards the location of a clot 2350. In some embodiments, it may be desirable to advance the second catheter 2340 through the first catheter 2320 to reach the clot 2350. In some embodiments, the clot 2350 may become engaged with or corded at a distal end of the second catheter. In such cases, it may be necessary to remove the second catheter 2340 from the vasculature of the patient to remove the clot 2350. As the second catheter 2340 is retracted from the first catheter 2320, debris from the stuck clot 2350 can dislodge. In such embodiments, application of vacuum at both the first and second catheters 2320, 2340 can beneficially prevent the debris from flowing distally into the vasculature of the patient by aspirating the debris and thus reducing the risk of embolization.

In some embodiments, the clot 2350 may become engaged with or corked at the distal end of the catheter 2320. Vacuum through the first catheter 2320 may prevent dislodgement of the clot 2350 from the catheter 2320. In some instances, it may not be readily apparent if portions of a clot are engaged with the catheter 2320 or the catheter 2340. In such instances, vacuum through both the catheters 2320 and 2340 may prevent debris from flowing distally into the vasculature of the patient by aspirating the debris and thus reducing the risk of embolization.

In some steps of a thrombectomy procedure, fluid flow rate along the first catheter 2320 and the aspiration line connected to the first catheter 2320 may be greater than a fluid flow rate along the second catheter 2340 and the aspiration line connected to the second catheter 2340 or may increase to a greater flow rate than in previous steps of the procedure. To reduce the fluid flow rate along the first catheter 2320 and the aspiration line connected to the first catheter 2320, a flow reducer (e.g., 1080, 1180, 1280, 1380, 1480, 1580, 1680, 1780, 1880, etc.) can be positioned along the flow path between the first catheter 2320 and the aspiration canister. The flow reducer can beneficially reduce the flow rate proximal to the flow reducer and thus reduce blood loss. The flow reducer can also maintain the fluid flow rate or reduce an amount of increase of the fluid flow rate proximal to the flow reducer when the second catheter 2340 is outside of the first catheter 2320, and the RHV of the first catheter is closed and/or the second aspiration line is closed in comparison to the increase in fluid flow rate that would occur in the absence of the flow reducer.

The flow reducer may also be configured to allow sufficient vacuum pressure to prevent dislodgement of the clot 2350 from the catheter 2320 in instances where the clot is engaged thereto, for example, when the second catheter 2340 is outside of the first catheter 2320 and the RHV is open and/or the second aspiration line is open.

Any of the embodiments described herein may be used with a clot filtering device, such as the embodiments shown and described in International Patent Application No. PCT/US2022/078113, filed Oct. 14, 2022, the contents of which are herein incorporated by reference in their entirety.

As used in the description and claims, the singular form “a”, “an” and “the” include both singular and plural references unless the context clearly dictates otherwise. For example, the term “vacuum line” may include, and is contemplated to include, a plurality of vacuum lines. At times, the claims and disclosure may include terms such as “a plurality,” “one or more,” or “at least one;” however, the absence of such terms is not intended to mean, and should not be interpreted to mean, that a plurality is not conceived.

In some embodiments, any of the RHVs, aspiration line configurations, and/or clot filtering devices may be used in manual thrombectomy procedures, in roboticized thrombectomy procedures, or in hybrid manual-automatic thrombectomy procedures. Various examples of automatic thrombectomy systems are shown and described in U.S. patent application Ser. No. 17/527,393 filed Nov. 16, 2021, the contents of which are herein incorporated by reference in their entirety.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-60. (canceled)

61. A method of performing a thrombectomy procedure, the method comprising:
connecting a distal end of a first aspiration line to a first catheter to establish a first fluid flow path between the first catheter and an aspiration canister, wherein a proximal end of the first aspiration line is coupled to a first port of the aspiration canister;
connecting a distal end of a second aspiration line to a second catheter to establish a second fluid flow path between the second catheter and the aspiration canister, wherein a proximal end of the second aspiration line is coupled to a second port of the aspiration canister;
advancing the first catheter and the second catheter though a vasculature of a patient to a target vascular site having a clot while the second catheter is positioned within a lumen of the first catheter;
applying uninterrupted vacuum to the aspiration canister to apply uninterrupted vacuum to the first catheter and the second catheter along the first fluid flow path and the second fluid flow path; and
retracting the second catheter out of a hemostasis valve of the first catheter while maintaining a vacuum pressure in the first catheter at or greater than a minimum vacuum pressure threshold for preventing debris from the clot from flowing distally into the vasculature of the patient.

62. The method of claim 61, wherein the minimum vacuum pressure threshold is −14 inHg.

63. The method of claim 62, wherein maintaining the vacuum pressure in the first catheter at or greater than the minimum vacuum pressure threshold comprises maintaining the vacuum pressure between −14 inHg and −28 inHg.

64. The method of claim 61, further comprising closing the hemostasis valve after retracting the second catheter out of the hemostasis valve and closing the second aspiration line.

65. The method of claim 64, further comprising maintaining a fluid flow rate along at least a portion of the first fluid flow path at or below a maximum threshold value when the hemostasis valve and the second aspiration line are closed, wherein the maximum threshold value of the fluid flow rate is between 140 ml/min and 200 mL/min.

66. The method of claim 64, wherein the vacuum pressure in the first catheter is maintained after retracting the second catheter out of the hemostasis valve and closing the second aspiration line.

67. The method of claim 64, wherein the vacuum pressure in the first catheter increases after retracting the second catheter out of the hemostasis valve and closing the second aspiration line.

68. The method of claim 61, wherein the first aspiration line comprises a clot retrieval device.

69. The method of claim 68, wherein the clot retrieval device comprises a distal port and a proximal port, the distal port having a diameter larger than a diameter of the proximal port.

70. The method of claim 68, wherein the first aspiration line comprises a first tubing section distal to the clot retrieval device and a second tubing section proximal to the clot retrieval device, wherein the second tubing section has an inner diameter smaller than an inner diameter of the first tubing section to reduce a fluid flow rate through the second tubing section.

71. The method of claim 68, wherein at least a portion of the clot retrieval device is transparent to allow clot visualization.

72. The method of claim 61, wherein each of the first port and the second port comprises a self-sealing port.

73. The method of claim 72, wherein the aspiration canister comprises a third port coupled to a vacuum source, wherein the third port has a different structure than the first port and the second port.

74. The method of claim 61, further comprising restricting the application of vacuum to the first catheter by closing a clamp positioned along the first aspiration line.

75. The method of claim 61, further comprising restricting the application of vacuum to the second catheter by closing a clamp positioned along the second aspiration line.

76. The method of claim 61, wherein retracting the second catheter out of the hemostasis valve of the first catheter further comprises maintaining a vacuum pressure in the second catheter at or greater than a minimum vacuum pressure threshold of the second catheter.

77. The method of claim 76, wherein the minimum vacuum pressure threshold of the second catheter is a minimum vacuum pressure threshold for preventing debris from the clot from flowing distally from the second catheter into the vasculature of the patient.

78. The method of claim 76, wherein the minimum vacuum pressure threshold of the second catheter is a minimum vacuum pressure threshold for maintaining the clot engaged to a distal tip of the second catheter.
